SEQUENCE LISTING (12 sequences)

SEQ ID NO: 1 - protein, 184 aa, Homo sapiens
MKLVSVALMYLGSLAFLGADTARLDVASEFRKKWNKWALSRGKRELRMSSYPTGLADVKAGPAQTLIRPQDMKGASRSPEDSSPDAARIRVKRYRQSMNNFQGLRSFGCRFGTCTVQKLAHQIYQFTDKDKDNVAPRSKISPQGYGRRRRRSLPEAGPGRTLVSSKPQAHGAPAPPSGSAPHFL

SEQ ID NO: 2 - protein, 164 aa, Homo sapiens
ARLDVASEFRKKWNKWALSRGKRELRMSSSYPTGLADVKAGPAQTLIRPQDMKGASRSPEDSSPDAARIRVKRYRQSMNNFQGLRSFGCRFGTCTVQKLAHQIYQFTDKDKDNVAPRSKISPQGYGRRRRRSLPEAGPGRTLVSSKPQAHGAPAPPSGSAPHFL

SEQ ID NO: 3 - protein, 21 aa, Homo sapiens
ARLDVASEFRKKWNKWALSRG

SEQ ID NO: 4 - protein, 20 aa, Homo sapiens
  MOD_RES 20: note = C-terminal amidation
ARLDVASEFRKKWNKWALSR

SEQ ID NO: 5 - protein, 48 aa, Homo sapiens
ELRMSSSYPTGLADVKAGPAQTLIRPQDMKGASRSPEDSSPDAARIRV

SEQ ID NO: 6 - protein, 53 aa, Homo sapiens
YRQSMNNFQGLRSFGCRFGTCTVQKLAHQIYQFTDKDKDNVAPRSKISPQGYG

SEQ ID NO: 7 - protein, 52 aa, Homo sapiens
  MOD_RES 52: note = C-terminal amidation
YRQSMNNFQGLRSFGCRFGTCTVQKLAHQIYQFTDKDKDNVAPRSKISPQGY

SEQ ID NO: 8 - protein, 38 aa, Homo sapiens
RRRRRSLPEAGPGRTLVSSKPQAHGAPAPPSGSAPHFL

SEQ ID NO: 9 - protein, 21 aa, Homo sapiens
YRQSMNNFQGLRSFGCRFGTC

SEQ ID NO: 10 - protein, 12 aa, Homo sapiens
CTVQKLAHQIYQ

SEQ ID NO: 11 - protein, 12 aa, Homo sapiens
  MOD_RES 12: note = C-terminal amidation
CAPRSKISPQGY

SEQ ID NO: 12 - protein, 13 aa, Homo sapiens
CAPRSKISPQGYG

(19) United States
(12) Patent Application Publication
(10) Pub. No.: US 2026/0063648 A1
(43) Pub. Date: Mar. 5, 2026

(54) Method for the prediction of a risk or severity of side effects in a cancer patient that is to be treated or has been treated with immune effector cell therapy (IECT)

(71) Applicant: PAM Theragnostics GmbH, Hennigsdorf (DE)

(72) Inventors: Yulia Ilina, Berlin (DE); Paul Kaufmann, Berlin (DE)

(73) Assignee: PAM Theragnostics GmbH, Hennigsdorf (DE)

(21) Appl. No.: 19/310,806

(22) Filed: Aug. 26, 2025

Related U.S. Application Data
(60) Provisional application No. 63/687,239, filed on Aug. 26, 2024.
(60) Provisional application No. 63/687,230, filed on Aug. 26, 2024.

(30) Foreign Application Priority Data
Aug. 26, 2024 (EP) 24196442.8
Aug. 26, 2024 (EP) 24196494.9

(51) Int. Cl. G01N 33/74

(52) U.S. Cl. CPC G01N 33/74; G01N 2333/575; G01N 2800/50; G01N 2800/52

(57) ABSTRACT

The present application is directed to a method for the prediction of a risk of side effects and/or prediction of the severity of side effects in a cancer patient that is to be treated or has been treated with immune effector cells in the frame of an immune effector cell therapy (IECT), and/or for assessing an effect of a treatment of side effects in a cancer patient that has been treated with immune effector cells in the frame of an IECT, the method comprising the steps of a) determining the level of at least one endothelial dysfunction biomarker in a sample of bodily fluid of the patient, and b) correlating the level of the at least one endothelial dysfunction biomarker in the sample to the risk of the side effects and/or the severity of the side effects in the patient and/or to the effect of the treatment of the side effects in the patient. Further aspects relate to corresponding methods stratification and medicaments for use in corresponding treatment.

### PRIORITY CLAIM

This application claims priority to U.S. Provisional Patent Application No. 63/687,230 filed on Aug. 26, 2024 and titled “ANTI-ADM ANTIBODIES, ANTI-ADM ANTIBODY FRAGMENT OR ANTI-ADM NON-IG SCAFFOLD FOR THERAPY OR PREVENTION OF IMMUNE EFFECTOR CELL THERAPY SIDE EFFECTS,” U.S. Provisional Patent Application No. 63/687,239 filed on Aug. 26, 2024 and titled “A METHOD FOR THE PREDICTION OF A RISK OR SEVERITY OF SIDE EFFECTS IN A CANCER PATIENT THAT IS TO BE TREATED OR HAS BEEN TREATED WITH IMMUNE EFFECTOR CELL THERAPY (IECT),” European Patent Application No. EP24196442 filed on Aug. 26, 2024 and titled “ANTI-ADM ANTIBODIES, ANTI-ADM ANTIBODY FRAGMENT OR ANTI-ADM NON-IG SCAFFOLD FOR THERAPY OR PREVENTION OF IMMUNE EFFECTOR CELL THERAPY SIDE EFFECTS,” and European Patent Application No. EP24196494 filed on Aug. 26, 2024 and titled “A METHOD FOR THE PREDICTION OF A RISK OR SEVERITY OF SIDE EFFECTS IN A CANCER PATIENT THAT IS TO BE TREATED OR HAS BEEN TREATED WITH IMMUNE EFFECTOR CELL THERAPY (IECT),” the entire contents of all of which are hereby incorporated by reference herein.

### SEQUENCE LISTING

The instant application contains a Sequence Listing which has been submitted electronically in XML format and is hereby incorporated by reference in its entirety. Said XML copy, created on Aug. 26, 2024, is named P75637EP.xml and is 12,854 bytes in size.

### FIELD OF THE INVENTION

The invention relates to a method for the prediction of a risk of side effects and/or prediction of the severity of side effects in a cancer patient that is to be treated or has been treated with immune effector cells in the frame of an immune effector cell therapy (IECT), and/or for assessing an effect of a treatment of side effects in a cancer patient that has been treated with immune effector cells in the frame of an immune effector cell therapy (IECT), the method comprising the steps of:
determining the level of at least one endothelial dysfunction biomarker in a sample of bodily fluid of said patient, and
correlating said level of said at least one endothelial dysfunction biomarker in said sample to the risk of said side effects and/or the severity of said side effects in said patient and/or to the effect of said treatment of said side effects in said patient.

The invention further relates to a method for prevention or treatment of side effects of immune effector cell therapy (IECT) in a cancer patient that is to be treated or has been treated with immune effector cells in the frame of an immune effector cell therapy (IECT), wherein the treatment is immunosuppressive therapy, wherein said patient is selected by a method comprising the steps:
determining at least one endothelial dysfunction biomarker in a sample of bodily fluid obtained from said patient and
correlating said level of at least one endothelial dysfunction biomarker in said sample to said risk of side effects, and
   wherein said side effects are selected from the group comprising cytokine release syndrome (CRS), and immune effector cell-associated neurotoxicity syndrome (ICANS).

The invention further relates to a medicament for use in the prevention or treatment of side effects of immune effector cell therapy (IECT) in a cancer patient that is to be treated or has been treated with immune effector cells in the frame of an immune effector cell therapy (IECT), wherein said patient is selected by a method comprising the steps:
determining at least one endothelial dysfunction biomarker in a sample of bodily fluid obtained from said patient and
correlating said level of at least one endothelial dysfunction biomarker in said sample to said risk of side effects, and
   wherein said side effects are selected from the group comprising cytokine release syndrome (CRS), and immune effector cell-associated neurotoxicity syndrome (ICANS), and wherein said medicament is an immunosuppressive medicament.

The invention further relates to a method for patient stratification and/or selection of a patient for treatment of side effects, wherein said patient is a cancer patient that is to be treated or has been treated with immune effector cells in the frame of an immune effector cell therapy (IECT), said method comprising:
determining the level of at least one endothelial dysfunction biomarker in a sample of bodily fluid of said patient and
assigning if said patient is to receive treatment of said side effects based on said level of at least one endothelial dysfunction biomarker in said sample.

### BACKGROUND

### (Pro-)Adrenomedullin

Adrenomedullin (ADM) is one of the best studied peptide hormones, which plays a role in a vast range of physiological and pathophysiological processes, including inter alia vasodilation, angiogenesis, and hormone regulation. ADM is also involved in bronchodilatation, renal function, cell growth, differentiation, neurotransmission, and modulation of the immune response. ADM belonging to the ADM/calcitonin gene-related peptide (CGRP) superfamily of peptides and is known to be produced in various human organs and tissues, including the heart, adrenal endothelial cells, lungs, kidneys, adipose tissue, and vascular endothelium, which contribute to ADM blood levels.

ADM mRNA encodes a preprohormone of 185 amino acids (SEQ ID NO: 1), the pre-pro-Adrenomedullin that is enzymatically converted into Proadrenomedullin by cleavage of the N-terminal signal peptide. Proadrenomedullin (SEQ ID NO: 2) is then further process by several prohormone convertases to result in four peptides, namely
PAMP-Gly (SEQ ID NO: 3): Proadrenomedullin N-terminal 20 peptide with a C-terminal glycine residue, inactive precursor of biologically active PAMP (PAMP-NH₂ (SEQ ID No. 4)),
MR-proADM (SEQ ID NO: 5): Mid-regional Proadrenomedullin, a stable and inert peptide,
ADM-Gly (SEQ ID NO: 6): C-terminally glycine extended, inactive precursor of biologically active ADM (mature ADM/ADM-NH₂ (SEQ ID NO: 7) and
CT-proADM (SEQ ID NO: 8): C-terminal Proadrenomedullin or Adrenotensin.

ADM-Gly is the direct, inactive biosynthetic precursor of the fully activated ADM form (mature ADM), often referred to as the intermediate form of ADM, and represents the dominating circulating form of ADM in humans. To gain its biological activity, ADM-Gly is activated by the Vitamin C dependent enzyme peptidylglycine-alpha amidating monooxygenase (PAM). PAM recognizes the C-terminal glycine and catalyses a sequential two-step reaction also referred to as amidation or C-terminal amidation.

Mature ADM has several physiological effects, such as vasodilation, angiogenesis, cardioprotection, nephroprotection, anti-oxidation, anti-apoptosis and tissue repair and regeneration. Mature ADM is involved in blood pressure regulation, bronchodilatation, renal function, hormone secretion, cell growth, differentiation, neurotransmission, and modulation of the immune response. Moreover, ADM plays a crucial role as autocrine factor during proliferation and regeneration of endothelial cells.

Additionally, mature ADM promotes angiogenesis, arteriogenesis, prevents cognitive decline after chronic cerebral hypoperfusion and is therefore considered as therapeutic agent in vascular dementia (reviewed in (Garcia et al., 2006) and (Bilint et al., 2023).

ADM is an effective vasodilator, and thus it is possible to associate the hypotensive effect with the particular peptide segments in the C-terminal part of ADM. It has furthermore been found that the above-mentioned physiologically active peptide PAMP formed from pre-proADM likewise exhibits a hypotensive effect, even if it appears to have an action mechanism differing from that of ADM (in addition to the above-mentioned review articles (Eto, 2001) and Hinson et al. 2000 see also Kuwasaki et al. 1997. FEBS Lett 414(1): 105-110: Kuwasaki et al. 1999. Ann. Clin. Biochem. 36: 622-628; Tsuruda et al. 2001 Life Sci. 69(2): 239-245 and EP-A2 0 622 458). It has furthermore been found that the concentrations of ADM, which can be measured in the circulation and other biological liquids, are in a number of pathological states, significantly above the concentrations found in healthy control subjects. Thus, the ADM level in patients with congestive heart failure, myocardial infarction, kidney diseases, hypertensive disorders, diabetes mellitus, in the acute phase of shock and in sepsis and septic shock are significantly increased, although to different extents. The PAMP concentrations are also increased in some of said pathological states, but the plasma levels are lower relative to ADM (Eto 2001. Peptides 22: 1693-1711). It was reported that unusually high concentrations of ADM are observed in sepsis, and the highest concentrations in septic shock (Eto 2001. Peptides 22: 1693-1711: Hirata et al. Journal of Clinical Endocrinology and Metabolism 81(4): 1449-1453: Ehlenz et al. 1997. Exp Clin Endocrinol Diabetes 105: 156-162: Tomoda et al. 2001. Peptides 22: 1783-1794: Ueda et al. 1999. Am. J. Respir. Crit. Care Med. 160: 132-136 and Wang et al. 2001. Peptides 22: 1835-1840). WO2004/097423 describes the use of an antibody against adrenomedullin for diagnosis, prognosis, and treatment of cardiovascular disorders. Treatment of diseases by blocking the ADM receptor are also described in the art, (e.g., WO2006/027147, PCT/EP2005/012844) said diseases may be sepsis, septic shock, cardiovascular diseases, infections, dermatological diseases, endocrinological diseases, metabolic diseases, gastroenterological diseases, cancer, inflammation, hematological diseases, respiratory diseases, muscle skeleton diseases, neurological diseases, urological diseases.

### Endothelial Dysfunction

Endothelial (barrier) dysfunction has been described for a number of diseases and is considered to contribute to the pathogenesis of these diseases. These include pulmonary diseases, ARDS occurring in COVID-19 and in asthma, arthritis, ulcerative colitis, other chronic inflammatory diseases, cancer, age-related macular degeneration, diabetic macular edema, psychiatric and neurodegenerative diseases, cardiovascular diseases, circulatory shock including septic shock, hepatic and renal diseases, infectious and autoimmune diseases, (Claesson-Welsh et al., 2021)(Rodrigues & Granger, 2015)(O Karpinich et al., 2011)(Soussi et al., 2023)(Opal & Van Der Poll, 2015)(W. L. Lee & Slutsky, 2010)(Chistiakov et al., 2015)(Claesson-Welsh, 2015).

### Endothelial Dysfunction and Adrenomedullin

Elevation of plasma ADM is widely accepted as a surrogate marker for disturbed endothelial barrier function. High ADM levels likely represent a failing compensatory response, aimed at restoring endothelial barrier function (van Lier et al., 2020). Elevated plasma concentrations have been described in most of the diseases, which are associated with endothelial dysfunction (see above).

### Other Endothelial Dysfunction Biomarkers

Endothelial dysfunction can be detected by quantifying soluble blood and cerebrospinal fluid (CSF) markers that are released into the circulation and offer a window into the diverse facets of endothelial function that contribute to homeostasis (Jaime Garcia et al., 2023); these include cell adhesion molecules (e.g. E-selectin, P-selectin, ICAM-1, VCAM-1), molecules involved in the coagulation pathway (e.g. von Willebrand factor (vWF) and soluble thrombomodulin), endoglin, endocan, cytokines, chemokines and pro-inflammatory markers (IL-6, CRP, LOX-1, CD40L, ADMA), proendothelin-1 or fragments thereof, soluble fms-like tyrosine kinase-1, matrix metalloproteinases (MMPs, e.g. MMP-7 and MMP-9), tissue inhibitor of metalloproteinases (TIMPs, e.g. TIMP-1) (Jaime Garcia et al., 2023)(Leite et al., 2020)(Zhang, 2022).

### Immune Effector Cell Therapy (IECT)

Over the past decades, an increasing number of ways to treat cancer has been developed (Link 2019). These include chemotherapy, hormone therapy, hyperthermia, photodynamic therapy, radiation therapy, stem cell transplant, surgery, targeted therapy, and immunotherapy. Recently, Immune Effector Cell Therapies (IECT) have emerged as the most promising therapy in oncology (Forero-Forero et al. 2021). Under the term IECT several types of cell therapies are summarized, which include Chimeric Antigen Receptor T-cell (CAR T) therapy, natural killer cell (NK) therapy, chimeric antigen receptor natural killer cell (CAR-NK) therapy, T cell receptor-engineered T cell (TCR T) therapy, tumor-infiltrating T cell (TIT)), and cytokine-induced killer cell (CIK) therapy, of which CAR T-cell therapy has been studied and used the most (Forero-Forero et al. 2021). CAR T-cell therapy has turned out to be particularly effective in the treatment of patients with B cell malignancies (Sun et al. 2024). Currently, CAR T-cell therapy is approved for the treatment of B cell relapsed or refractory leukemia and lymphoma, and most recently, multiple myeloma. In these different diseases, it has led to excellent complete and overall response rates depending on the patient population and therapy. The field of CAR T-cell therapy continues to expand rapidly. IECTs other than CAR T, including CAR-NK, TIL, CIK are being explored in hematologic malignancies and solid malignancies such as germ cell tumor, sarcoma, neuro-blastoma, and melanoma (Kanate et al. 2023).

Despite promising efficacy, CAR T cell therapy as well as the other IECTs mentioned above are frequently associated with significant side effects (Lee et al. 2019). The two most notable toxicities are cytokine release syndrome (CRS) and immune effector cell-associated neurotoxicity syndrome (ICANS) (Chohan, Siegler, and Kenderian 2023).

CRS has been defined as “a supraphysiologic response following any immune therapy that results in the activation or engagement of endogenous or infused T cells and/or other immune effector cells. Symptoms can be progressive, must include fever at the onset, and may include hypotension, capillary leak (hypoxia) and end organ dysfunction. (Lee et al. 2019)”.

ICANS has been defined as “a disorder characterized by a pathologic process involving the central nervous system following any immune therapy that results in the activation or engagement of endogenous or infused T cells and/or other immune effector cells. Symptoms or signs can be progressive and may include aphasia, altered level of consciousness, impairment of cognitive skills, motor weakness, seizures, and cerebral edema (Lee et al. 2019)”. Similar to CRS, ICANS should be applied to any immune effector cell engaging therapy, not just CAR T-cells.

CRS is one of the most common side effects associated with CAR T-cell therapy with an incidence of 20-50% and manifests as fever and difficulty of breathing, low blood pressure, nausea and vomiting, and poses a notable safety challenge and can lead to life-threatening conditions, such as hypotension, respiratory distress and organ insufficiency (Sun et al. 2024).

Regimes have been developed to treat CAR T-cell-induced toxicity. These include supportive care, the use of tocilizumab, an IL-6 receptor antibody, and steroids, if patients are refractory to tocilizumab. Even with the use of tocilizumab, which is FDA approved to treat severe CRS, severe CRS and death still occur.

For the treatment of neurotoxicity, tocilizumab is not effective, and steroids are used as first line of treatment there. The pathophysiology and mechanisms underlying neurotoxicity are only barely understood (Neelapu et al. 2018).

Other agents which have been investigated and are generally reserved for refractory CRS include anti-IL-6 antibody (siltuximab), anti-TNF antibody (etanercept, infliximab) and anti-IL-1 antibody (anakinra). They all aim at downregulating the overactivation of the immune system.

Due to the side-effects of IECTs and the so far limited possibilities to treat these, experts have called for novel toxicity-directed therapies and low-toxicity constructs which do not compromise treatment efficacy, which are desperately needed (Chohan, Siegler, and Kenderian 2023).

The immune system emerges as a key player not only mediating cytokine responses but potentially contributing to endothelial injury in CAR T-cell toxicity (Gavriilaki et al. 2020). Thus, endothelial dysfunction has been investigated in CAR T-cell toxicity (Sumransub et al. 2022). Following CAR T-cell therapy, endothelial dysfunction characterized by increased pro-inflammatory signaling (e.g., IL-6, IL-8, MCP-1) loss of barrier function has been observed, and CAR T-cells have been shown to directly induce endothelial dysfunction (Rosen et al. 2023).

The administration of CAR T-cells is preceded by a chemotherapy to achieve lymphodepletion, which is applied typically for three days, and subsequently, after a rest of two days the CAR T-cells are infused. One study has been published, in which the influence of chemotherapy on the levels of Adrenomedullin was investigated (GGler et al. 2006). The study was restricted to pediatric patients and did not comprise a following CAR T-cell therapy. The authors reported slightly higher levels of Adrenomedullin after chemotherapy compared to baseline (30.3 pmol/mL vs 25.1 pmol/mL).

At this point, there are no publications describing biomarker status prior to immune effector cell administration (in particular CAR T-cell administration) in the frame of an immune effector cell therapy (in particular in the frame of a CAR T-cell therapy) or shortly after administration of immune effector cells (in particular CAR T-cells) in the frame of an immune effector cell therapy (in particular in the frame of a CAR T-cell therapy), which would predict an increased risk for endothelial dysfunction, which would develop as a side effect of immune effector cell therapy (in particular CAR T-cell therapy), nor how to prevent such side-effect.

After side effects, i.e. CRS, have occurred and treatment of side effects has been initiated, there is then during the course of treatment uncertainty about when CRS is considered resolved (Lee et al. 2019).

Under the state of the art, once such therapies are administered, the patient is considered to still have CRS, even in the absence of fever, until all signs and symptoms leading to the diagnosis of CRS are considered resolved, which puts additional burden, e.g. on medical personnel and on the patient due to excessive need for patient surveillance. Thus, there is the need for better tools to monitor the success of treatment of side effects resulting from IECTs.

It is the surprising finding of the present invention that prior to or after immune effector cell administration in the frame of an immune effector cell therapy, endothelial dysfunction, as reflected by increased proADM concentrations (e.g. ADM-Gly or bio-ADM concentrations), occurs in patients developing side effects of said therapy, but not in those who do not develop side effects. Therefore, the determination of the level of proADM or fragments thereof (e.g. ADM-Gly or bio-ADM) can be used in a method for the prediction of a risk of side effects as well as the prediction of the severity of side effects in a cancer patient that is to be treated or has been treated with immune effector cells in the frame of an immune effector cell therapy (IECT). Moreover, the determination of the level of proADM or fragments thereof (e.g. ADM-Gly or bio-ADM) can be used in a method for assessing an effect of a treatment of side effects in a cancer patient that has been treated with immune effector cells in the frame of an immune effector cell therapy (IECT).

### Plausibility Other ED Markers

As ADM-Gly and bio-ADM are markers of endothelial dysfunction and are shown to be applicable as markers prediction of a risk of side effects and/or prediction of the severity of side effects in a cancer patient that is to be treated or has been treated with immune effector cells in the frame of an immune effector cell therapy (IECT), and/or for assessing an effect of a treatment of side effects in a cancer patient that has been treated with immune effector cells in the frame of an immune effector cell therapy (IECT), it is plausible that other biomarkers known to be involved in endothelial dysfunction, may be used as alternative biomarkers.

### DESCRIPTION OF THE INVENTION

Subject matter of the present invention is a method for the prediction of a risk of side effects and/or prediction of the severity of side effects in a cancer patient that is to be treated or has been treated with immune effector cells in the frame of an immune effector cell therapy (IECT), and/or for assessing an effect of a treatment of side effects in a cancer patient that has been treated with immune effector cells in the frame of an immune effector cell therapy (IECT), the method comprising the steps of:
determining the level of at least one endothelial dysfunction biomarker in a sample of bodily fluid of said patient, and
correlating said level of said at least one endothelial dysfunction biomarker in said sample to the risk of said side effects and/or the severity of said side effects in said patient and/or to the effect of said treatment of said side effects in said patient.

A further subject matter of the present invention is a method for prevention or treatment of side effects of immune effector cell therapy (IECT) in a cancer patient that is to be treated or has been treated with immune effector cells in the frame of an immune effector cell therapy (IECT), wherein the treatment is immunosuppressive therapy, wherein said patient is selected by a method comprising the steps:
determining at least one endothelial dysfunction biomarker in a sample of bodily fluid obtained from said patient and
correlating said level of at least one endothelial dysfunction biomarker in said sample to said risk of side effects, and
   wherein said side effects are selected from the group comprising cytokine release syndrome (CRS), and immune effector cell-associated neurotoxicity syndrome (ICANS).

Another subject matter of the present invention is a medicament for use in the prevention or treatment of side effects of immune effector cell therapy (IECT) in a cancer patient that is to be treated or has been treated with immune effector cells in the frame of an immune effector cell therapy (IECT), wherein said patient is selected by a method comprising the steps:
determining at least one endothelial dysfunction biomarker in a sample of bodily fluid obtained from said patient and
correlating said level of at least one endothelial dysfunction biomarker in said sample to said risk of side effects, and
   wherein said side effects are selected from the group comprising cytokine release syndrome (CRS), and immune effector cell-associated neurotoxicity syndrome (ICANS), and wherein said medicament is an immunosuppressive medicament.

Another subject matter of the present invention is A method for patient stratification and/or selection of a patient for treatment of side effects, wherein said patient is a cancer patient that is to be treated or has been treated with immune effector cells in the frame of an immune effector cell therapy (IECT), said method comprising:
determining the level of at least one endothelial dysfunction biomarker in a sample of bodily fluid of said patient and
assigning if said patient is to receive treatment of said side effects based on said level of at least one endothelial dysfunction biomarker in said sample.

### DEFINITIONS AND EMBODIMENTS

The term “prediction of a risk” in the context of the present invention denotes a prediction (assignment of a probability) of how a patient's medical condition will progress. This may include an estimation of the chance of recovery or the chance (risk) of an adverse outcome (e.g., side effects due to a treatment) for said patient. In specific embodiments the patient may preferably be accounted to a certain risk category, wherein categories comprise for instance high risk versus low risk, or risk categories based on numeral values, such as risk category 1, 2, 3, etc.

The stratified patient groups may include patients that require an initiation of treatment and patients that do not require initiation of treatment.

Thus, the present invention is also a method for patient stratification and/or selection of a patient for treatment of side effects, wherein said patient is a cancer patient that is to be treated or has been treated with immune effector cells in the frame of an immune effector cell therapy (IECT), said method comprising:
determining the level of at least one endothelial dysfunction biomarker in a sample of bodily fluid of said patient and
assigning if said patient is to receive treatment of said side effects based on said level of at least one endothelial dysfunction biomarker in said sample.

The term “immune effector cell therapy (IECT)” summarizes cell therapies in the field of oncology including Chimeric Antigen Receptor T-cell (CAR-T) therapy, natural killer cell (NK) therapy, chimeric antigen receptor natural killer cell (CAR-NK) therapy, T cell receptor-engineered T-cell (TCR T) therapy, tumor-infiltrating T cell (TIT)), and cytokine-induced killer cell (CIK) therapy.

In one embodiment of the invention said immune effector cell therapy (IECT) is selected from the group comprising Chimeric Antigen Receptor T-cell (CAR-T) therapy, natural killer cell (NK) therapy, chimeric antigen receptor natural killer cell (CAR-NK) therapy, T cell receptor-engineered T-cell (TCR T) therapy, tumor-infiltrating T cell (TIT)), and cytokine-induced killer cell (CIK) therapy.

In a specific embodiment of the invention said immune effector cell therapy (IECT) is Chimeric Antigen Receptor T-cell (CAR-T) therapy Side effects of immune effector cell therapy (IECT) are for example cytokine release syndrome (CRS) and immune effector cell-associated neurotoxicity syndrome (ICANS).

CRS is defined as a supraphysiologic response following any immune therapy that results in the activation or engagement of endogenous or infused T cells and/or other immune effector cells. Symptoms can be progressive, must include fever at the onset, and may include hypotension, capillary leak (hypoxia) and end organ dysfunction (D. W. Lee et al., 2019).

ICANS is defined as a disorder characterized by a pathologic process involving the central nervous system following any immune therapy that results in the activation or engagement of endogenous or infused T cells and/or other immune effector cells. Symptoms or signs can be progressive and may include aphasia, altered level of consciousness, impairment of cognitive skills, motor weakness, seizures, and cerebral edema (D. W. Lee et al., 2019).

In one embodiment of the invention said side effects of immune effector cell therapy (IECT) may be selected from the group comprising cytokine release syndrome (CRS) and immune effector cell-associated neurotoxicity syndrome (ICANS).

The severity of side effects is classified into different grades of cytokine release syndrome (CRS) or immune effector cell-associated neurotoxicity syndrome (ICANS). According to guidelines, grade 1 is mild, grade 2 is moderate, grade 3 is severe, grade 4 is life-threatening and grade 5 leads to death (D. W. Lee et al., 2019).

In one embodiment of the invention a severity of side effects is predicted, wherein said side effects are cytokine release syndrome (CRS) or immune effector cell-associated neurotoxicity syndrome (ICANS) and the severity is mild (grade 1), moderate (grade 2), severe (3) or life-threatening (grade 4) or leads to death (grade 5).

The term “assessing an effect of a treatment of side effects” refers to the response of said patient to said treatment of side effects, in particular a worsening or the recovery from said side effects.

Particular embodiments of the method of the invention as further detailed herein relate to the prediction of a risk of side effects in a cancer patient that is to be treated with immune effector cells in the frame of an immune effector cell therapy (IECT) and assessing an effect of a treatment of side effects in said patient after said patient has been treated with said immune effector cells.

Other particular embodiments of the method of the invention as further detailed herein relate to the prediction of a risk of side effects and assessing an effect of a treatment of side effects in a cancer patient that has been treated with immune effector cells in the frame of an immune effector cell therapy (IECT).

Other particular embodiments of the method of the invention as further detailed herein relate to the prediction of the severity of side effects in a cancer patient that is to be treated with immune effector cells in the frame of an immune effector cell therapy (IECT) and assessing an effect of a treatment of side effects in said patient after said patient has been treated with said immune effector cells.

Other particular embodiments of the method of the invention as further detailed herein relate to the prediction of the severity of side effects in a cancer patient that has been treated with immune effector cells in the frame of an immune effector cell therapy (IECT) and assessing an effect of a treatment of side effects in said patient after said patient has been treated with said immune effector cells.

More particular embodiments of the method of the invention as further detailed herein relate to the prediction of a risk of side effects in a cancer patient that is to be treated with immune effector cells in the frame of an immune effector cell therapy (IECT).

Other more particular embodiments of the method of the invention as further detailed herein relate to the prediction of a risk of side effects in a cancer patient that has been treated with immune effector cells in the frame of an immune effector cell therapy (IECT).

More particular embodiments of the method of the invention as further detailed herein relate to the prediction of the severity of side effects in a cancer patient that is to be treated with immune effector cells in the frame of an immune effector cell therapy (IECT).

Other more particular embodiments of the method of the invention as further detailed herein relate to the prediction of the severity side effects in a cancer patient that has been treated with immune effector cells in the frame of an immune effector cell therapy (IECT).

Other more particular embodiments of the method of the invention as further detailed herein relate to assessing an effect of a treatment of side effects in a cancer patient that has been treated with immune effector cells in the frame of an immune effector cell therapy (IECT).

In one embodiment of the invention said determination of the level of at least one endothelial dysfunction biomarker and said correlation is performed more than once in said patient. This refers to controlling the development (detection of any changes) of a disease and or pathophysiological condition of a patient, e.g. risk or severity of a disease or condition or assessment of an effect to a treatment.

In a specific embodiment of the invention said determination of the level of at least one endothelial dysfunction biomarker and said correlation is performed more than once in said patient, wherein particularly said determination of the level of at least one endothelial dysfunction biomarker and said correlation is performed once before administration of immune effector cells to said patient and one or more times after administration of immune effector cells to said patient; or alternatively said determination of the level of at least one endothelial dysfunction biomarker and said correlation is performed at least twice after administration of immune effector cells to said patient.

In one embodiment of the invention said treatment of side effects is administered if an increased risk of said side effects is predicted for said patient and/or said patient is in need of adjustment of said treatment of side effects if said treatment of side effects is being determined as not effective.

In the context of the present invention, said adjustment of treatment of side effects in said patient, in particular the adjustment of immunosuppressive treatment drugs can involve initiation and/or a change and/or withdrawal of said treatment, in particular the initiation and/or change and/or withdrawal of said immunosuppressive treatment drugs. Said adjustment of treatment drugs may be a change in the dose, the administration route or regime or other parameters of medication treatment, in particular immunosuppressive medication treatment. Furthermore, an adjustment in the treatment with, in particular immunosuppressive drugs may also and potentially additionally relate to a change in the one or more drugs used for treating the patient. In some embodiments, a change can therefore relate to the replacement of one or more, in particular immunosuppressive drugs by one or more other agents. In a very specific embodiment of the invention said immunosuppressive treatment drug is withdrawn from the patient.

In a specific embodiment assessing the effect of a treatment of side effects may mean the assessment of the need of adjustment of treatment of side effects, in particular treatment with immunosuppressive drugs in said patient using measurement of at least one endothelial dysfunction biomarker (e.g. proADM or fragments thereof).

In one embodiment of the invention said treatment of side effects is selected from the group comprising antihistamines, non-steroidal anti-inflammatory drugs (NSAIDs), immunosuppressive therapy, vasopressors, fluids and supportive oxygen supply.

In a specific embodiment of the invention said immunosuppressive therapy is selected from the group comprising anti-IL-6 antibody (e.g. siltuximab), anti-IL-6 receptor antibody (e.g. tocilizumab), anti-TNF-antibody (e.g. etanercept, infliximab), anti-IL-1-antibody (e.g. anakinra), and/or corticosteroids.

Corticosteroids may be selected from the group consisting of glucocorticoids or mineralocorticoids. Glucocorticoids may be selected from the group comprising cortisone, hydrocortisone, prednisone, prednisolone, methylprednisolone, dexamethasone or betamethasone. Mineralocorticoids may be selected from the group comprising fludrocortisone.

In a specific embodiment of the invention said corticosteroid is selected from the group comprising cortisone, hydrocortisone, prednisone, prednisolone, methylprednisolone, dexamethasone, betamethasone or fludrocortisone.

In one embodiment of the invention said endothelial dysfunction biomarker is selected from the group comprising Proadrenomedullin (proADM) or fragments thereof, adhesion molecules (e.g. E-selectin, P-selectin, ICAM-1, VCAM-1), molecules involved in the coagulation pathway (e.g. von Willebrand factor (vWF) and soluble thrombomodulin), endoglin, endocan, IL-6, CRP, LOX-1, CD40L, ADMA, proendothelin-1 or fragments thereof (in particular ET-1, Big-ET, NT-proET-1), matrix metalloproteinases (MMPs, e.g. MMP-7 and MMP-9), and tissue inhibitor of metalloproteinases (TIMPs, e.g. TIMP-1).

In a more specific embodiment of the invention said endothelial dysfunction biomarker is selected from the group comprising proADM or fragments thereof, adhesion molecules (e.g. E-selectin, P-selectin, ICAM-1, VCAM-1), molecules involved in the coagulation pathway (e.g. von Willebrand factor (vWF) and soluble thrombomodulin), endoglin, endocan, proendothelin-1 or fragments thereof (in particular ET-1, Big-ET, NT-proET-1), matrix metalloproteinases (MMPs, e.g. MMP-7 and MMP-9), and tissue inhibitor of metalloproteinases (TIMPs, e.g. TIMP-1).

In a specific embodiment of the invention said endothelial dysfunction biomarker is proADM (SEQ ID NO. 2) or a fragment thereof.

Said fragment of pro-Adrenomedullin (proADM) is selected from the group comprising, PAMP-Gly (SEQ ID NO: 3), mature PAMP (SEQ ID NO: 4), MR-proADM (SEQ ID NO: 5), ADM-Gly (SEQ ID NO: 6), mature ADM (SEQ ID NO: 7) and CT-proADM (SEQ ID NO: 8).

Mature ADM, bio-ADM and ADM-NH₂ is used synonymously throughout this application and is a molecule according to SEQ ID No.: 7.

In the embodiments of the invention, said threshold is in particular a predetermined threshold.

In embodiments of the present invention if said level of at least one endothelial dysfunction biomarker is above the threshold, the patient has an increased risk of developing the side effects. In embodiments of the present invention if said level of at least one endothelial dysfunction biomarker is below the threshold, the patient has a decreased risk of developing the side effects.

In embodiments of the present invention if said level of at least one endothelial dysfunction biomarker is above the threshold, the patient has an increased risk of developing more severe side effects. In embodiments of the present invention if said level of at least one endothelial dysfunction biomarker is below the threshold, the patient has a decreased risk of developing more severe side effects.

In embodiments of the present invention if said level of at least one endothelial dysfunction biomarker is above the threshold, the treatment is not effective. In embodiments of the present invention if said level of at least one endothelial dysfunction biomarker is below the threshold, the treatment is effective.

In embodiments of the present invention if said level of at least one endothelial dysfunction biomarker is above a previously determined level of at least one endothelial dysfunction biomarker in a sample of bodily fluid of said patient, said treatment is not effective. In embodiments of the present invention if said level of at least one endothelial dysfunction biomarker is below a previously determined level of at least one endothelial dysfunction biomarker in a sample of bodily fluid of said patient, said treatment is effective.

In embodiments of the present invention if said level of at least one endothelial dysfunction biomarker is above the threshold, the patient is assigned as having an increased risk of developing the side effects. In embodiments of the present invention if said level of at least one endothelial dysfunction biomarker is below the threshold, the patient is assigned as having a decreased risk of developing the side effects.

In embodiments of the present invention if said level of at least one endothelial dysfunction biomarker is above the threshold, the treatment is assigned as not being effective. In embodiments of the present invention if said level of at least one endothelial dysfunction biomarker is below the threshold, the treatment is assigned as being effective.

In embodiments of the present invention if said level of at least one endothelial dysfunction biomarker is above a previously determined level of at least one endothelial dysfunction biomarker in a sample of bodily fluid of said patient, said treatment is assigned as being not effective. In embodiments of the present invention if said level of at least one endothelial dysfunction biomarker is below a previously determined level of at least one endothelial dysfunction biomarker in a sample of bodily fluid of said patient, said treatment is assigned as being effective.

In embodiments of the present invention if said level of at least one endothelial dysfunction biomarker is above a previously determined level of at least one endothelial dysfunction biomarker in a sample of bodily fluid of said patient, said patient is at risk of developing said side effects and said medicament is to be administered to said patient. In embodiments of the present invention if said level of at least one endothelial dysfunction biomarker is below a previously determined level of at least one endothelial dysfunction biomarker in a sample of bodily fluid of said patient, said patient is not at risk of developing said side effects and said medicament is not to be administered to said patient.

In embodiments of the present invention if said level of at least one endothelial dysfunction biomarker is above a previously determined level of at least one endothelial dysfunction biomarker in a sample of bodily fluid of said patient, said patient is assigned as being at risk of developing said side effects and said medicament is to be administered to said patient. In embodiments of the present invention if said level of at least one endothelial dysfunction biomarker is below a previously determined level of at least one endothelial dysfunction biomarker in a sample of bodily fluid of said patient, said patient is assigned as being not at risk of developing said side effects and said medicament is not to be administered to said patient.

In embodiments of the present invention if said level of at least one endothelial dysfunction biomarker is above a previously determined level of at least one endothelial dysfunction biomarker in a sample of bodily fluid of said patient, treatment for said side effects is administered to said patient. In embodiments of the present invention if said level of at least one endothelial dysfunction biomarker is below a previously determined level of at least one endothelial dysfunction biomarker in a sample of bodily fluid of said patient, treatment for said side effects is not administered to said patient.

In embodiments of the present invention if said level of at least one endothelial dysfunction biomarker is above a previously determined level of at least one endothelial dysfunction biomarker in a sample of bodily fluid of said patient, said patient is assigned to receive treatment of said side effects. In embodiments of the present invention if said level of at least one endothelial dysfunction biomarker is below a previously determined level of at least one endothelial dysfunction biomarker in a sample of bodily fluid of said patient, said patient is assigned not to receive treatment of said side effects.

In one embodiment of the invention said fragment of Pro-Adrenomedullin is mature ADM (SEQ ID NO. 7) and the threshold of the level of mature ADM in a sample of bodily fluid of said patient is between 35 and 125 pg/ml, more preferred between 40 and 100 pg/ml, even more preferred between 50 and 90 pg/ml, most preferred said threshold is 70 pg/ml.

In a further particular embodiment of the invention said fragment of Pro-Adrenomedullin is mature ADM and said threshold is an x-fold of the median level of mature ADM in a healthy population, in particular in the range between the 2.6-fold and 9.1-fold, more particular in the range between the 2.9-fold and 7.3-fold, more particular in the range between 3.6-fold and 6.6-fold, most particular said threshold is the 5.1-fold of the median of the level of mature ADM in a healthy population.

In one embodiment of the invention said fragment of Pro-Adrenomedullin is ADM-Gly (SEQ ID No. 6) and the threshold of the level of ADM-Gly in a sample of bodily fluid of said patient is between 25 and 125 pg/ml, more preferred between 30 and 100 pg/ml, even more preferred between 35 and 75 pg/ml, most preferred said threshold is 40 pg/ml.

In a further particular embodiment of the invention said fragment of Pro-Adrenomedullin is ADM-Gly and said threshold is an x-fold of the median level of ADM-Gly in a healthy population, in particular in the range between the 0.9-fold and 4.6-fold, more particular in the range between the 1.1-fold and 3.7-fold, more particular in the range between 1.3-fold and 2.8-fold, most particular said threshold is the 1.5-fold of the median of the level of ADM-Gly in a healthy population.

In one embodiment of the invention said fragment of Pro-Adrenomedullin is MR-proADM (SEQ ID No. 5) and the threshold of the level of MR-proADM in a sample of bodily fluid of said patient is between 0.5 and 2 nmol/L, more preferred between 0.6 and 1.5 nmol/L, even more preferred between 0.7 and 1 nmol/L, most preferred said threshold is 0.8 nmol/L.

In further particular embodiments of the invention said fragment of Pro-Adrenomedullin is MR-proADM and the threshold is an x-fold of the median level of MR-proADM in a healthy population, in particular in the range between 1.2-fold and 4.9-fold, more preferred between 1.5-fold and 3.7-fold, even more preferred between 1.7-fold and 2.4-fold, most preferred said threshold is 2.0-fold of the median of the level of MR-proADM in a healthy population.

In one embodiment of the invention said fragment of Pro-Adrenomedullin is mature PAMP (SEQ ID NO. 4) and the threshold of the level of mature PAMP in a sample of bodily fluid of said patient is between 0.7 and 1.2 pmol/L, more preferred between 0.8 and 1.0 pmol/L, most preferred said threshold is 0.9 pmol/L.

In further particular embodiments of the invention said fragment of Pro-Adrenomedullin is mature PAMP and said threshold is an x-fold of the mean level of mature PAMP in a healthy population, in particular in the range between the 1.4-fold and 2.4-fold, more particular in the range between 1.6-fold and 2.0-fold, most particular said threshold is the 1.8-fold of the mean of the level of mature PAMP in a healthy population.

In one embodiment of the invention said fragment of Pro-Adrenomedullin is PAMP-Gly (SEQ ID No. 3) and the threshold of the level of PAMP-Gly in a sample of bodily fluid of said patient is between 1.5 and 2.4 pmol/L, even more preferred between 1.8 and 2.2 pmol/L, most preferred said threshold is 2 pmol/L and 2.4 pmol/L, even more preferred between 1.8 and 2.2 pmol/L, most preferred said threshold is 2 pmol/L.

In further particular embodiments of the invention said fragment of Pro-Adrenomedullin is PAMP-Gly and said threshold is an x-fold of the mean level of PAMP-Gly in a healthy population, in particular in the range between the 1.3-fold and 2.1-fold, more particular in the range between 1.6-fold and 1.9-fold, most particular said threshold is the 1.7-fold of the mean of the level of PAMP-Gly in a healthy population.

The above-mentioned threshold values might show a certain amount of variation, depending on the assay used to determine the level of the respective endothelial dysfunction biomarker (e.g. pro-ADM or fragment thereof), if these have been calibrated differently from the assay systems used in the present invention. Therefore, the above-mentioned thresholds shall apply in particular for the assay systems used in the present invention, and shall apply for such differently calibrated assays accordingly, taking into account the differences in calibration. One possibility of quantifying the difference in calibration is a method comparison analysis (correlation) of the assay in question (e.g. proADM or fragments thereof) with the respective biomarker assay used in the present invention by measuring the respective biomarker (pro-ADM or a fragment thereof) in samples using both assays. Assuming a linear correlation between the assays, another possibility is to determine with the assay in question, given this test has sufficient analytical sensitivity, the median biomarker level of a representative normal population, compare results with the median biomarker levels as described in the literature (e.g. bio-ADM: Weber et al. 2017. J Appl Lab Med 2(2): 222-233; MR-proADM: Smith et al. 2009. Clin Chem 55:1593-1595; PAMP-Gly: Hashida et al. 2004. Clinical Biochemistry 37 14-21; mature PAMP: Hashida et al. 2004. Clinical Biochemistry 37 14-21) and recalculate the calibration based on the difference obtained by this comparison, e.g. by applying a factor. Hence, the x-fold of e.g. the mean or median (or a specific percentile) of the level of Pro-Adrenomedullin or a fragment thereof in a healthy population can be used as a threshold level using a differently calibrated assay, taking into account the above precautions.

Methods to quantify fragments derived from proADM have been described, e.g. the measurement of mature ADM (Weber et al. 2017. J Appl Lab Med 2(2): 222-233), MR-proADM (Morgenthaler et al. 2005. Clin Chem 51(10):1823-9), mature PAMP (Hashida et al. 2004. Clinical Biochemistry 37 14-21), PAMP-Gly (Hashida et al. 2004. Clinical Biochemistry 37 14-21) and CT-proADM (EP 2 111 552). For example, a commercial homogeneous time-resolved fluoroimmunoassay for the measurement of MR-proADM in plasma on a fully automated system is available (BRAHMS MR-proADM KRYPTOR; BRAHMS GmbH, Hennigsdorf, Germany) (Caruhel et al. 2009. Clin Biochem 42(7-8):725-8).

In certain embodiments the level of CT-proADM is determined by a sandwich immunoassay, wherein an anti-CT-proADM antibody is immobilized on a surface, a liquid comprising CT-proADM and a second, labeled anti-CT-proADM antibody is added, and after a washing step, the binding of the second antibody is measured based on detection of the label, e.g. by determining chemiluminescence.

The plasma median concentration of mature ADM (bio-ADM) in a normal (healthy) population was 13.7 pg/ml, the lowest value 11 pg/ml and the 99^{th} percentile 43 pg/ml (Weber et al. 2017. J Appl Lab Med 2(2): 222-233).

The plasma median MR-proADM concentration in normal (healthy) subjects was 0.41 (interquartile range 0.23-0.64) nmol/L (Smith et al. 2009. Clin Chem 55:1593-1595) using the automated sandwich fluorescence assay for the detection of MR-proADM as described in Caruhel et al. (Caruhel et al. 2009. Clin Biochem 42:725-8).

The plasma median concentration of CT-proADM in normal healthy subjects (n=200) was 77.6 pmol/L (min 46.6 pmol/L, max 136.2 pmol/L) and the 95% percentile was 113.8 pmol/L (EP 2 111 552 B1).

The plasma mean concentration of PAMP-Gly in normal healthy subjects (n=51) was 1.15 pmol/L+/−0.38 pmol/L (Hashida et al. 2004. Clinical Biochemistry 37: 14-21).

The plasma mean concentration of mature PAMP in normal healthy subjects (n=51) was 0.51 pmol/L+/−0.19 pmol/L (Hashida et al. 2004. Clinical Biochemistry 37: 14-21).

The threshold level is a level, which allows for allocating the patient into a group of patients who have been diagnosed and/or are having an increased risk of an adverse event (e.g., mortality), or into a group of patients who have not been diagnosed and/or have a decreased risk of an adverse event, or into a severity group. Thus, the threshold level shall allow for differentiating between a patient who is diagnosed and/or is having an increased risk of an adverse event and a patient who is not diagnosed and/or is having a decreased risk of an adverse event.

It is known in the art how threshold levels can be determined. Threshold levels are predetermined values and are set to meet routine requirements in terms of, e.g., specificity and/or sensitivity. These requirements can vary. It may for example be that sensitivity or specificity, respectively, has to be set to certain limits, e.g., 80%, 90%, 95% or 98%, respectively.

The sensitivity and specificity of a diagnostic and/or prognostic test depends on more than just the analytical “quality” of the test, they also depend on the definition of what constitutes an abnormal result. In practice, Receiver Operating Characteristic curves (ROC curves), are typically calculated by plotting the value of a variable versus its relative frequency in “reference group” (i.e. patients who do not have the disease) and “disease” populations (i.e. patients who have the disease). For any particular marker, a distribution of marker levels for patients with and without disease will likely overlap. Under such conditions, a test does not absolutely distinguish patients with and without disease (e.g., endothelial dysfunction) with 100% accuracy, and the area of overlap indicates where the test cannot distinguish normal from disease. A threshold is selected, above which (or below which, depending on how a marker changes with the disease) the test is considered to be abnormal and below which the test is considered to be normal. The area under the ROC curve is a measure of the probability that the perceived measurement will allow correct identification of a condition. ROC curves can be used even when test results do not necessarily give an accurate number. As long as one can rank results, one can create a ROC curve. For example, results of a test on “disease” samples might be ranked according to degree (e.g. 1=low, 2=normal, and 3=high). This ranking can be correlated to results in the “reference” group, and a ROC curve created. These methods are well known in the art (See, e.g., Hanley et al. 1982. Radiology 143: 29-36). Preferably, ROC curves result in an Area under the ROC curve (AUC) of greater than about 0.5, more preferably greater than about 0.7, still more preferably greater than about 0.8, even more preferably greater than about 0.85, and most preferably greater than about 0.9. The term “about” in this context refers to +/−5% of a given measurement.

The horizontal axis of the ROC curve represents (one minus specificity), which increases with the rate of false positives. The vertical axis of the curve represents sensitivity, which increases with the rate of true positives. Thus, for a particular cut-off threshold selected, the value of (one minus specificity) may be determined, and a corresponding sensitivity may be obtained. The area under the ROC curve is a measure of the probability that the measured marker level will allow correct identification of a disease or condition. Thus, the area under the ROC curve can be used to determine the effectiveness of the test.

In addition to the normal range, other methods may be used to determine thresholds for a specific indication, depending on the intended use and application/clinical setting. Such methods include e.g. the Youden optimum, thresholds that maximise overall accuracy, the odds ratio, or the positive or negative predictive value. In some situations, thresholds achieving a pre-specified level of sensitivity or specificity (e.g. 80%, 90%, 95% or 99%) can be appropriate for the clinical application. The choice of methods depends on the clinical application, which weights the costs of false positive and false negative results based on the test result consequences for the patient and the health care system, as well as the clinical need. Finally, multiple approaches may be combined to define a consensus threshold.

A “reference group” may be a healthy population, e.g., with no signs and symptoms of a disease. A reference group may consist of more than one reference subjects.

Particular threshold values are for instance the 90th, 95th or 99th percentile of a reference group (e.g. healthy population). By using a higher percentile than the 75th percentile, one reduces the number of false positive subjects identified, but one might miss to identify subjects, who are at moderate, albeit still increased risk. Thus, one might adopt the threshold value depending on whether it is considered more appropriate to identify most of the subjects at risk at the expense of also identifying “false positives”, or whether it is considered more appropriate to identify mainly the subjects at high risk at the expense of missing several subjects at moderate risk.

For example, the 75^{th} percentile, more particular the 90^{th} percentile, even more particular a 95^{th} percentile, most particular the 99^{th} percentile values can be used for the upper limits of the normal range.

Biomarkers can also be used for the prediction of a future event or risk (e.g. prediction of a risk of side effects). For time-to-event data threshold levels can be obtained for instance from a Kaplan-Meier analysis, where the occurrence of a disease is correlated with e.g. the tertiles, quartiles, quintiles of the endothelial dysfunction marker (e.g. proADM or fragments thereof) in the population. There are also equivalent methods available to the ROC methods described before, based on i.e. time-dependent ROC analysis or generalizations of the area under the ROC curve (C index).

The threshold level may vary depending on various physiological parameters such as age, gender or sub-population, as well as on the means used for the determination of the at least one endothelial dysfunction biomarker (e.g. Pro-Adrenomedullin and fragments thereof) referred to herein.

In one embodiment of the present invention the level of at least one endothelial dysfunction biomarker (e.g. proADM or fragments thereof) is determined by contacting said sample of bodily fluid with a capture binder that binds specifically to said at least one endothelial dysfunction biomarker (e.g. proADM or fragments thereof).

In a specific embodiment, said capture-binder exhibits a binding affinity to said at least one endothelial dysfunction biomarker (e.g. proADM or fragments thereof) of at least 10⁷ M⁻¹, preferred 10⁸ M⁻¹, more preferred affinity is greater than 10⁹ M⁻¹, most preferred greater than 10¹⁰ M⁻¹. A person skilled in the art knows that it may be considered to compensate lower affinity by applying a higher dose of compounds and this measure would not lead out-of-the-scope of the invention.

In one embodiment, said capture-binder is an antibody binding to at least one endothelial dysfunction biomarker (e.g. proADM or fragments thereof).

To determine the affinity of the antibodies to at least one endothelial dysfunction biomarker (e.g. proADM or fragments thereof), in one embodiment the kinetics of binding of an endothelial dysfunction biomarker to an immobilized antibody are determined by means of label-free surface plasmon resonance using a Biacore 2000 system (GE Healthcare Europe GmbH, Freiburg, Germany). Reversible immobilization of the antibodies was performed using an anti-mouse Fe antibody covalently coupled in high density to a CM5 sensor surface according to the manufacturer's instructions (mouse antibody capture kit; GE Healthcare), (Lorenz et al. 2011. Antimicrob Agents Chemother. 55 (1): 165-173).

Alternatively, the at least one endothelial dysfunction biomarker (e.g. proADM or fragments thereof) bound to a capture binder on a solid phase is detected with a second capture binder specifically binding to said at least one endothelial dysfunction biomarker (e.g. proADM or fragments thereof).

In one embodiment such assay for determining the level of at least one endothelial dysfunction biomarker (e.g. proADM or fragments thereof) is a sandwich immunoassay using any kind of detection technology including but not restricted to enzyme label, chemiluminescence label, electrochemiluminescence label, preferably a fully automated assay. In one embodiment of the diagnostic method such an assay is an enzyme labeled sandwich assay. Examples of automated or fully automated assay comprise assays that may be used for one of the following systems: Roche Elecsys®, Abbott Architect®, Siemens Advia Centauer®, Siemens Immulite®, Brahms Kryptor®, Biomerieux Vidas®, Alere Triage®, Boditech AFIAS®, Ortho Vidas®, Diasorin LIASION®, Beckman Dxl®, Lumira Dx®, MeMed Key®, Werfen BioFlash®, BioRad BioPlex®.

A variety of immunoassays are known and may be used for the assays and methods of the present invention. In one embodiment of the present invention, the immunoassay is selected from the group, luminescence immunoassay (LIA), immunoluminometric assay (ILMA), radioimmunoassays (“RIA”), homogeneous enzyme-multiplied immunoassays (“EMIT”), enzyme linked immunoabsorbent assays (“ELISA”), apoenzyme reactivation immunoassay (“ARIS”), chemiluminescence—(“CLIA”), electrochemiluminescence—(“ECLIA”) and fluorescence-immunoassays, luminescence-based bead arrays, magnetic beads based arrays, protein microarray assays, rapid test formats such as for instance dipstick immunoassays, immuno-chromatographic strip tests, rare cryptate assay and automated systems/analyzers.

In one embodiment of the invention the assay or method may be a so-called POC (point-of-care)-test that is a test technology, which allows performing the test within less than 1 hour near the patient without the requirement of a fully automated assay system. One example for this technology is the immunochromatographic test technology, e.g., a microfluidic device.

In a specific embodiment at least one of said two binders is labeled in said sandwich immunoassay in order to be detected.

In another preferred embodiment said label is selected from the group comprising chemiluminescent label, enzyme label, fluorescence label, radioiodine label.

The assays can be homogenous or heterogeneous assays, competitive and non-competitive assays. In one embodiment, the assay is in the form of a sandwich assay, which is a non-competitive immunoassay, wherein the molecule to be detected and/or quantified is bound to a first antibody and to a second antibody. The first antibody may be bound to a solid phase, e.g. a bead, a surface of a well or other container, a chip or a strip, and the second antibody is an antibody which is labeled, e.g. with a dye, with a radioisotope, or a reactive or catalytically active moiety. The amount of labeled antibody bound to the analyte is then measured by an appropriate method. The general composition and procedures involved with “sandwich assays” are well-established and known to the skilled person (The Immunoassay Handbook, Ed. David Wild, Elsevier LTD, Oxford: 3rd ed. (May 2005), ISBN-13: 978-0080445267; Hultschig C et al., Curr Opin Chem Biol. 2006 February; 10(1):4-10. PMID: 16376134).

In another embodiment the assay comprises two capture molecules, preferably antibodies which are both present as dispersions in a liquid reaction mixture, wherein a first labelling component is attached to the first capture molecule, wherein said first labelling component is part of a labelling system based on fluorescence- or chemiluminescence-quenching or amplification, and a second labelling component of said marking system is attached to the second capture molecule, so that upon binding of both capture molecules to the analyte a measurable signal is generated that allows for the detection of the formed sandwich complexes in the solution comprising the sample.

In another embodiment, said labeling system comprises rare earth cryptates or rare earth chelates in combination with fluorescence dye or chemiluminescence dye, in particular a dye of the cyanine type. In an embodiment of the present invention, fluorescence-based assays comprise the use of dyes, are selected from the group comprising FAM (5- or 6-carboxyfluorescein), VIC, NED, Fluorescein, Fluoresceinisothiocyanate (FITC), IRD-700/800, Cyanine dyes, such as CY3, CY5, CY3.5, CY5.5, Cy7, Xanthen, 6-Carboxy-2′,4′,7′,4,7-hexachlorofluorescein (HEX), TET, 6-Carboxy-4′,5′-dichloro-2′,7′-dimethoxyfluorescein (JOE), N,N,N′,N′-Tetramethyl-6-carboxyrhodamine (TAMRA), 6-Carboxy-X-rhodamine (ROX), 5-Carboxyrhodamine-6G (R6G5), 6-carboxyrhodamine-6G (RG6), Rhodamine, Rhodamine Green, Rhodamine Red, Rhodamine 110, BODIPY dyes, such as BODIPY TMR, Oregon Green, Coumarins such as Umbelliferone, Benzamides, such as Hoechst 33258; Phenanthridines, such as Texas Red, Yakima Yellow, Alexa Fluor, PET, Ethidium bromide, Acridinium dyes, Carbazol dyes, Phenoxazine dyes, Porphyrine dyes, Polymethine dyes, and the like.

In the context of the present invention, chemiluminescence based assays comprise the use of dyes, based on the physical principles described for chemiluminescent materials in (Kirk-Othmer, Encyclopedia of chemical technology, 4th ed., executive editor, J. I. Kroschwitz; editor, M Howe-Grant, John Wiley & Sons, 1993, vol. 15, p. 518-562, incorporated herein by reference, including citations on pages 551-562). Preferred chemiluminescent dyes are acridinium esters.

As mentioned herein, an “assay” or “diagnostic assay” can be of any type applied in the field of diagnostics.

Such an assay may be based on the binding of an analyte to be detected to one or more capture probes with a certain affinity. Concerning the interaction between capture molecules and target molecules or molecules of interest, in one embodiment the affinity constant is preferably greater than 10⁸ M⁻¹.

Alternatively, the level of any of the above analytes may be determined by other analytical methods e.g. mass spectroscopy.

In another embodiment of the present invention the at least one endothelial dysfunction biomarker (e.g. proADM or fragments thereof) is determined using a point-of-care (POC) device.

In another preferred embodiment of invention said point-of-care device is a microfluidic device.

As used herein, a microfluidic device has a plurality of chambers arranged at different positions which are connected in parallel and into which a fixed amount of fluid may be efficiently distributed without using a separate driving source, wherein said device includes a platform having a center of rotation and including at least one microfluidic structure. Microfluidic devices are used to perform biological or chemical reactions by manipulating small amounts of fluid.

In a preferred embodiment, the bio-ADM level is determined from a plasma sample of said patient. It is however typical in the technical lifecycle improvement of measurement of analytes that possibilities exist to measure such analytes in other—in particular blood-based—matrices. For instance, in case of bio-ADM, another technology has been developed, which uses whole (EDTA-) blood, known as IB10 Sphingotest® bio-ADM (https://www.nexus-dx.com/wp-content/uploads/2020/07/bio-ADM-IFU-REV-A.pdf). The IB10 Sphingotest® bio-ADM® is a rapid point-of-care (POC) immunoassay for the in vitro quantitative determination of human amidated adrenomedullin peptide (1-52), in the following referred to as bioactive adrenomedullin (bio-ADM®), in human EDTA whole blood and plasma.

In one embodiment the analytical assay sensitivity of said assay for ADM-Gly is able to quantify ADM-Gly of healthy subjects and is 20 pg/ml, preferably 15 pg/ml and more preferably 10 pg/ml.

In one embodiment the analytical assay sensitivity of said assay for ADM-Gly is able to quantify ADM-Gly of healthy subjects and is a 0.7-fold, preferably a 0.6-fold and more preferably 0.4-fold of the median of a healthy population.

In one embodiment the analytical assay sensitivity of said assay for PAMP is able to quantify PAMP of healthy subjects and is <0.5 pmol/L, preferably <0.25 pmol/L and more preferably <0.1 pmol/L.

In one embodiment the analytical assay sensitivity of said assay for PAMP is able to quantify PAMP of healthy subjects and is a 1.0-fold, preferably a 0.5-fold and more preferably a 0.2-fold of the mean of a healthy population.

In one embodiment the analytical assay sensitivity of said assay for the detection of CT-proADM is able to quantify CT-proADM of healthy subjects and is <100 pmol/L, preferably <75 pmol/L and more preferably <50 pmol/L.

In one embodiment the analytical assay sensitivity of said assay for the detection of CT-proADM is able to quantify CT-proADM of healthy subjects and is a 1.3-fold, preferably a 1.0-fold and more preferably a 0.6-fold of the median of a healthy population.

In one embodiment the analytical assay sensitivity of said assay for the detection of mature ADM is able to quantify mature of healthy subjects and is <40 pg/ml, preferably <25 pg/ml and more preferably <10 pg/ml.

In one embodiment the analytical assay sensitivity of said assay for the detection of mature ADM is able to quantify mature of healthy subjects and is a 2.9-fold, preferably a 1.8-fold and more preferably a 0.7-fold of the median of a healthy population.

In one embodiment the analytical assay sensitivity of said assay is able to quantify MR-proADM of healthy subjects and is <0.5 nmol/L, preferably <0.4 nmol/L and more preferably <0.2 nmol/L.

In one embodiment the analytical assay sensitivity of said assay is able to quantify MR-proADM of healthy subjects and is a 1.2-fold, preferably a 1.0-fold and more preferably a 0.5-fold of the median of a healthy population.

In one embodiment of the method described herein, the method additionally comprises comparing the determined level of said at least one endothelial dysfunction biomarker (e.g. proADM or fragments thereof) to a reference and/or threshold level, wherein said comparing is carried out in a computer processor using computer executable code.

The methods of the present invention may in part be computer-implemented. For example, the step of comparing the detected level of at least one endothelial dysfunction biomarker, e.g., proADM or fragments thereof, with a reference and/or threshold level can be performed in a computer system. For example, the determined values may be entered (either manually by a health professional or automatically from the device(s) in which the respective marker level(s) has/have been determined) into the computer-system. The computer-system can be directly at the point-of-care (e.g., primary care unit or ED) or it can be at a remote location connected via a computer network (e.g., via the internet, or specialized medical cloud-systems, optionally combinable with other IT-systems or platforms such as hospital information systems (HIS)). Alternatively, or in addition, the associated therapy guidance and/or therapy stratification will be displayed and/or printed for the user (typically a health professional such as a physician).

A bodily fluid according to the present invention is in one particular embodiment a blood sample. In one embodiment of the invention said blood sample may be selected from the group comprising whole blood, serum and plasma. In a specific embodiment of the method said sample is selected from the group comprising human citrate plasma, heparin plasma and EDTA plasma.

In embodiments of the present invention, “patient that is to be treated with immune effector cells in the frame of an immune effector cell therapy (IECT)” means in particular that the patient is in the time span between lymphodepletion and administration of the immune effector cells, more particularly within 14 days, or within 7 days, or within 2 days before said CAR T-cells are administered to said patient, even more particularly within 48 hours, or within 24 hours, or within 12 hours, or within 6, or within 2 hours, or within 1 hour before said immune effector cells are administered to said patient. This also means in particular that said method for the prediction of a risk of side effects is carried out in said patient before said immune effector cells are administered, more particularly in the time span between lymphodepletion and administration of the immune effector cells, more particularly within 14 days, or within 7 days, or within 2 days before said immune effector cells are administered to said patient, even more particularly within 48 hours, or within 24 hours, or within 12 hours, or within 6, or within 2 hours, or within 1 hour before said immune effector cells are administered to said patient.

In embodiments of the present invention, “patient that has been treated with immune effector cells in the frame of an immune effector cell therapy (IECT)” means in particular that the patient has received said treatment but has not developed side effects from said treatment at the time-point of taking the sample of bodily fluid.

In embodiments of the present invention, “patient that has been treated with immune effector cells in the frame of an immune effector cell therapy (IECT)” means in particular within 1 week, or within 2 days after said immune effector cells are administered to said patient, even more particularly within 48 hours, or within 24 hours, or within 12 hours, or within 6, or within or within 3 hours after said immune effector cells are administered to said patient. This means in particular embodiments that said method for the prediction of a risk of side effects is carried out in said patient after said immune effector cells are administered, more particularly within 1 week, or within 2 days after said immune effector cells are administered to said patient, even more particularly within 48 hours, or within 24 hours, or within 12 hours, or within 6, or within 3 hours after said immune effector cells are administered to said patient. This means in other particular embodiments that said assessing is carried out in said patient after said immune effector cells are administered, more particularly within 1 week, or within 2 days after said immune effector cells are administered to said patient, even more particularly within 48 hours, or within 24 hours, or within 12 hours, or within 6, or within 3 hours after said immune effector cells are administered to said patient.

In the embodiments of the invention, said side effects are in particular side effects of said treatment with immune effector cells.

In embodiments of the present invention the side effect is a side effect occurring upon administration of the immune effector cells, in particular related to administration immune effector cells. Such a side effect may in particular embodiments occur within 1 week after the administration of the immune effector cells to said patient. In other particular embodiments such a side effect may occur within 48 hours, more particular within 24 hours, even more particular within 12 hours, even more particular within 6 hours, even more particular within 3 hours after the administration of the immune effector cells to said patient.

In embodiments of the present invention said determination of the level of at least one endothelial dysfunction biomarker and said correlation is performed more than once in said patient, said determination and correlation are in particular once a day, more particularly once every 12 hours, even more particularly or once every hour.

For sake of completeness, the further embodiments of the invention detailed herein likewise read on the aspect of the invention relating to a method for prevention or treatment of side effects of immune effector cell therapy (IECT) in a cancer patient that is to be treated or has been treated with immune effector cells in the frame of an immune effector cell therapy (IECT), wherein the treatment is immunosuppressive therapy, wherein said patient is selected by a method comprising the steps:
determining at least one endothelial dysfunction biomarker in a sample of bodily fluid obtained from said patient and
correlating said level of at least one endothelial dysfunction biomarker in said sample to said risk of side effects, and
   wherein said side effects are selected from the group comprising cytokine release syndrome (CRS), and immune effector cell-associated neurotoxicity syndrome (ICANS),
   such as in particular embodiment relating to, the severity of side effects, specific immune effector cell therapies, specific endothelial dysfunction biomarkers and/or biomarker thresholds, specific samples of bodily fluid, time points and/or repetitions of determination of the level of endothelial dysfunction biomarkers, specific medicaments, such as immunosuppressive therapies, and antibodies.

For sake of completeness, the further embodiments of the invention detailed herein likewise read on the aspect of the invention relating to a method for patient stratification and/or selection of a patient for treatment of side effects, wherein said patient is a cancer patient that is to be treated or has been treated with immune effector cells in the frame of an immune effector cell therapy (IECT), said method comprising:
determining the level of at least one endothelial dysfunction biomarker in a sample of bodily fluid of said patient and
assigning if said patient is to receive treatment of said side effects based on said level of at least one endothelial dysfunction biomarker in said sample,
   such as in particular embodiment relating to specific side effects, the severity of side effects, specific immune effector cell therapies, specific endothelial dysfunction biomarkers and/or biomarker thresholds, specific samples of bodily fluid, time points and/or repetitions of determination of the level of endothelial dysfunction biomarkers, specific medicaments, such as immunosuppressive therapies, and antibodies.

For sake of completeness, the further embodiments of the invention detailed herein likewise read on the aspect of the invention relating to a medicament for use in the prevention or treatment of side effects of immune effector cell therapy (IECT) in a cancer patient that is to be treated or has been treated with immune effector cells in the frame of an immune effector cell therapy (IECT), wherein said patient is selected by a method comprising the steps:
determining at least one endothelial dysfunction biomarker in a sample of bodily fluid obtained from said patient and
correlating said level of at least one endothelial dysfunction biomarker in said sample to said risk of side effects, and
   wherein said side effects are selected from the group comprising cytokine release syndrome (CRS), and immune effector cell-associated neurotoxicity syndrome (ICANS), and wherein said medicament is an immunosuppressive medicament,
   such as in particular embodiment relating to specific side effects, the severity of side effects, specific immune effector cell therapies, specific endothelial dysfunction biomarkers and/or biomarker thresholds, specific samples of bodily fluid, time points and/or repetitions of determination of the level of endothelial dysfunction biomarkers, and antibodies.

Throughout the specification the “antibodies”, or “antibody fragments” or “non-Ig scaffolds” in accordance with the invention are capable to bind at least one endothelial dysfunction biomarker (e.g. proADM or fragments thereof), and thus are directed against at least one endothelial dysfunction biomarker (e.g. proADM or fragments thereof).

The term “antibody” generally comprises monoclonal and polyclonal antibodies and binding fragments thereof, in particular Fc-fragments as well as so called “single-chain-antibodies” (Bird et al. 1988), chimeric, humanized, in particular CDR-grafted antibodies, and dia or tetrabodies (Holliger et al. 1993). Also comprised are immunoglobulin-like proteins that are selected through techniques including, for example, phage display to specifically bind to the molecule of interest contained in a sample. In this context the term “specific binding” refers to antibodies raised against the molecule of interest or a fragment thereof. An antibody is considered to be specific, if its affinity towards the molecule of interest or the aforementioned fragment thereof is at least preferably 50-fold higher, more preferably 100-fold higher, most preferably at least 1000-fold higher than towards other molecules comprised in a sample containing the molecule of interest. It is well known in the art how to make antibodies and to select antibodies with a given specificity.

An antibody or fragment according to the present invention is a protein including one or more polypeptides substantially encoded by immunoglobulin genes that specifically binds an antigen. The recognized immunoglobulin genes include the kappa, lambda, alpha (IgA), gamma (IgG₁, IgG₂, IgG₃, IgG₄), delta (IgD), epsilon (IgE) and mu (IgM) constant region genes, as well as the myriad immunoglobulin variable region genes. Full-length immunoglobulin light chains are generally about 25 Kd or 214 amino acids in length.

Full-length immunoglobulin heavy chains are generally about 50 Kd or 446 amino acid in length. Light chains are encoded by a variable region gene at the NH₂-terminus (about 110 amino acids in length) and a kappa or lambda constant region gene at the COOH-terminus. Heavy chains are similarly encoded by a variable region gene (about 116 amino acids in length) and one of the other constant region genes.

The basic structural unit of an antibody is generally a tetramer that consists of two identical pairs of immunoglobulin chains, each pair having one light and one heavy chain. In each pair, the light and heavy chain variable regions bind to an antigen, and the constant regions mediate effector functions. Immunoglobulins also exist in a variety of other forms including, for example, Fv, Fab, and (Fab′)₂, as well as bifunctional hybrid antibodies and single chains (e.g., Lanzavecchia et al. 1987. Eur. J. Immunol. 17:105: Huston et al. 1988. Proc. Natl. Acad. Sci. U.S.A., 85:5879-5883: Bird et al. 1988. Science 242:423-426; Hood et al. 1984, Immunology, Benjamin, N.Y, 2nd ed.; Hunkapiller and Hood 1986 Nature 323:15-16). An immunoglobulin light or heavy chain variable region includes a framework region interrupted by three hypervariable regions, also called complementarity determining regions (CDR's) (see, Sequences of Proteins of Immunological Interest, E. Kabat et al. 1983, U.S. Department of Health and Human Services). As noted above, the CDRs are primarily responsible for binding to an epitope of an antigen. An immune complex is an antibody, such as a monoclonal antibody, chimeric antibody, humanized antibody or human antibody, or functional antibody fragment, specifically bound to the antigen.

Chimeric antibodies are antibodies whose light and heavy chain genes have been constructed, typically by genetic engineering, from immunoglobulin variable and constant region genes belonging to different species. For example, the variable segments of the genes from a mouse monoclonal antibody can be joined to human constant segments, such as kappa and gamma 1 or gamma 3. In one example, a therapeutic chimeric antibody is thus a hybrid protein composed of the variable or antigen-binding domain from a mouse antibody and the constant or effector domain from a human antibody, although other mammalian species can be used, or the variable region can be produced by molecular techniques. Methods of making chimeric antibodies are well known in the art, e.g., see U.S. Pat. No. 5,807,715.

A “humanized” immunoglobulin is an immunoglobulin including a human framework region and one or more CDRs from a non-human (such as a mouse, rat, or synthetic) immunoglobulin. The non-human immunoglobulin providing the CDRs is termed a “donor” and the human immunoglobulin providing the framework is termed an “acceptor.” In one embodiment, all the CDRs are from the donor immunoglobulin in a humanized immunoglobulin. Constant regions need not be present, but if they are, they must be substantially identical to human immunoglobulin constant regions, i.e., at least about 85-90%, such as about 95% or more identical. Hence, all parts of a humanized immunoglobulin, except possibly the CDRs, are substantially identical to corresponding parts of natural human immunoglobulin sequences. A “humanized antibody” is an antibody comprising a humanized light chain and a humanized heavy chain immunoglobulin. A humanized antibody binds to the same antigen as the donor antibody that provides the CDR's. The acceptor framework of a humanized immunoglobulin or antibody may have a limited number of substitutions by amino acids taken from the donor framework. Humanized or other monoclonal antibodies can have additional conservative amino acid substitutions, which have substantially no effect on antigen binding or other immunoglobulin functions. Exemplary conservative substitutions are those such as gly, ala; val, ile, leu; asp, glu; asn, gln; ser, thr; lys, arg; and phe, tyr.

Humanized immunoglobulins can be constructed by means of genetic engineering (e.g., see U.S. Pat. No. 5,585,089). Human antibodies can also be prepared by using transgenic animals carrying a human immunoglobulin gene (for example, see WO 93/12227; WO 91/10741).

A human antibody is an antibody wherein the light and heavy chain genes are of human origin. Human antibodies can be generated using methods known in the art.

Antibodies can be produced by immortalizing a (e.g. mammalian) B cell secreting the antibody of interest. Immortalization can be accomplished, for example, by EBV infection of a B-cell or by fusing a B cell with a myeloma to make a hybridoma or fusing a B cell to a hybridoma cell to produce a trioma cell. Antibodies can also be produced by phage display methods (see, e.g., WO 91/17271; WO 92/001047; WO 92/20791) or selected from a combinatorial monoclonal antibody library (see the Morphosys website).

### Methods for Obtaining Monoclonal Antibodies

In all of the following embodiments, the term monoclonal antibody is meant to include monoclonal antibodies, as well as fragments of monoclonal antibodies, such as the ones detailed herein, more particularly monoclonal antibodies.

### Hybridoma

In a further aspect, the antibody according to the present invention is a monoclonal antibody obtainable by a method comprising:
i) fusing antibody-secreting cells from an animal previously immunized with an antigen with myeloma cells to obtain a multitude of hybridomas,
ii) isolating from said multitude of hybridomas a hybridoma producing a desired monoclonal antibody.

In certain embodiments, the antibody according to the present invention is a monoclonal antibody obtainable by isolating from a multitude of hybridomas a hybridoma producing a desired monoclonal antibody, wherein said multitude of hybridomas were produced by fusing antibody-secreting cells from an animal previously immunized with an antigen with myeloma cells to obtain multitude of hybridomas.

A desired monoclonal antibody is in particular a monoclonal antibody binding the antigen, in particular with a binding affinity of at least 10⁷ M⁻¹, preferred 10⁸ M⁻¹, more preferred affinity is greater than 10⁹ M⁻¹, most preferred greater than 10¹⁰ M⁻¹.

In certain embodiments of the method for obtaining an antibody, in step i) the animal is a mammal, particularly a rabbit, a mouse or a rat, more particularly a mouse, more particularly a Balb/c mouse.

In certain embodiments of the method for obtaining an antibody, in step i) the antibody-secreting cell is a splenocyte, more particularly an activated B-cell.

In certain embodiments of the method for obtaining an antibody, in step i) fusing involves the use of polyethylene glycol.

In certain embodiments of the method for obtaining an antibody, in step i) the myeloma is derived from a mammal, in certain embodiments from the same species of mammal from which the multitude of antibody-secreting cells is obtained. In certain specific embodiments of the method for obtaining an antibody, in step i) the myeloma cells are of the cell line SP2/0.

In certain embodiments of the method for obtaining an antibody, said fusing in step i) comprises PEG-assisted fusion, Sendai virus-assisted fusion or electric current-assisted fusion.

In certain embodiments of the method for obtaining an antibody, said isolating in step ii) comprises performing an antibody capture assay, an antigen capture assay, and/or a functional screen.

In certain embodiments of the method for obtaining an antibody, in step ii) isolating the hybridoma producing a desired monoclonal antibody may involve cloning and re-cloning the hybridomas using the limiting-dilution technique.

### In One Embodiment, Said Antigen Capture Assay Comprises:

a) binding the produced antibodies to a substrate, particularly a solid substrate,
b) allowing antigen to bind to said antibodies,
c) removing unbound antigen by washing,
d) detecting bound antigen;

### Or Said Antigen Capture Assay Comprises:

a) allowing an antigen to bind the produced antibodies to form an antibody-antigen complex,
b) binding said antibody-antigen complex to a substrate, particularly a solid substrate,
c) removing unbound antigen by washing,
d) detecting bound antigen.

In one embodiment, said isolating of step ii) comprises performing an enzyme-linked immunosorbent assay, fluorescence-activated cell sorting, cell staining, immunoprecipitation, and/or a western blot. In one embodiment, said detecting of the antibody or the antigen is accomplished with an immunoassay.

In one embodiment, the animal is a transgenic animal, in particular a transgenic mouse (wherein in particular the mouse immunoglobulin (Ig) gene loci have been replaced with human loci within the transgenic animal genome), such as HuMabMouse or XenoMouse.

In one embodiment, the antigen comprises a peptide as described herein in Table 1, which in certain embodiments (in particular for immunization) may be conjugated to a protein, particularly a serum protein, more particularly a serum albumin, more particularly BSA.

In a preferred embodiment, the antibody according to the present invention is a monoclonal antibody obtainable by a method comprising:
i) fusing splenocytes cells from a Balb/c mouse previously immunized with a peptide as described herein in Table 1 with SP2/0 myeloma cells using polyethylene glycol, to obtain a multitude of hybridomas,
ii) isolating from said multitude of hybridomas a hybridoma producing a desired monoclonal antibody;

### More Preferably, the Method Comprises:

1) growing hybridomas for a first period (in particular 2 weeks) in HAT medium [RPMI 1640 culture medium supplemented with 20% fetal calf serum and HAT-Supplement]
2) followed replacing HAT medium with HT Medium for a multitude of passages (in particular 3)
3) followed by returning to the normal cell culture medium for a second time period, in particular until the end of three weeks after fusion
4) primary screening of cell culture supernatants for antigen-specific IgG antibodies
5) propagating microcultures of cells that tested positive in 4)
6) retesting cell culture supernatants of microcultures for antigen-specific IgG antibodies
7) cloning and re-cloning cultures that tested positive in 6), using the limiting-dilution technique
8) optionally determining the isotypes of clones obtained from 7)
9) optionally purifying antibodies via Protein A

### Phage Display

In a further aspect, the antibody according to the present invention is a monoclonal antibody obtainable by a method comprising:
i) isolating at least one antibody having affinity to an antigen from an antibody gene library;
ii) generating at least one cell strain expressing said at least one antibody;
iii) isolating the at least one antibody from a culture of the at least one cell strain obtained in step ii).

An antibody having affinity to an antigen is in particular an antibody with a binding affinity of at least 10⁷ M⁻¹, preferred 10⁸ M⁻¹, more preferred affinity is greater than 10⁹ M⁻¹, most preferred greater than 10¹⁰ M⁻¹.

In a certain embodiment, the antibody according to the present invention is a monoclonal antibody obtainable by isolating at least one antibody from a culture derived from at least one cell strain which expressed at least one antibody having affinity to an antigen from an antibody gene library.

In one embodiment, the antigen comprises a peptide as described herein in Table 1, which in certain embodiments may be bound to a solid phase.

In certain embodiments of the method for obtaining an antibody, in step i) the antibody gene library is a naive antibody gene library, particularly a human naive antibody gene library, more particularly in said library the antibodies are presented via phage display, i.e. on phages comprising a nucleotide sequence encoding for such respective antibody; more particularly the library HAL 7, HAL 8, or HAL 9, more particularly a library comprising the human naive antibody gene libraries HAL7/8.

In certain embodiments of the method for obtaining an antibody, in step i) screening comprises the use of an antigen, particularly an antigen containing a tag, more particularly a biotin tag, linked thereto via two different spacers. In particular embodiments, such panning strategy includes a mix of panning rounds with non-specifically bound antigen and antigen bound specifically via the tag, in the case of a biotin tag, bound to streptavidin. In this way, the background of non-specific binders may be minimized.

In certain embodiments of the method for obtaining an antibody, in step i), in embodiments wherein the library is a phage display library, the antibody is isolated by isolating a phage presenting said antibody (and comprising a nucleotide sequence encoding for the antibody).

In certain embodiments of the method for obtaining an antibody, in step ii) said cell strain is generated via introduction of a nucleotide sequence encoding for the antibody), in embodiments wherein the library in step i) is a phage display library, the isolated phage from step i) may be used to produce a bacterial strain, e.g. an E. coli strain, expressing the antibody.

In certain embodiments of the method for obtaining an antibody, in step iv); in embodiments wherein the library in step i) is a phage display library and wherein a bacterial strain is produced in step ii), antibody may be isolated from the supernatant of the culture.

It is understood that, as used in describing the methods for obtaining an antibody, the term “one antibody” in the expression “at least one antibody” in particular may include more than one antibody molecule of antibodies having the same amino acid sequence. This understanding applies, mutatis mutandis, to the term “one cell strain”.

In certain embodiments of the method for obtaining an antibody, more than one antibody (referring to a multitude of antibodies having distinct amino acid sequences, respectively) is isolated in step i) and accordingly more than one cell strain is generated in step ii). Such method may involve the selection of clones that are positive for binding to the antigen, e.g. via a binding assay, e.g. an ELISA assay involving the antigen, and cells positive for binding to the antigen may be isolated to produce monoclonal cell strains.

In a preferred embodiment, the antibody according to the present invention is a monoclonal antibody obtainable by a method comprising:
i) isolating at least one antibody having affinity to an antigen from an antibody gene library comprising the human naive antibody gene libraries HAL7/8, by eluting phages carrying said antibody from the library;
ii) generating at least one E. coli cell strain expressing said at least one antibody;
iii) isolating the at least one antibody from the supernatant a culture of the at least one E. coli cell strain obtained in step ii).

In a further aspect, an antibody fragment according to the present invention is produced by a method in volving enzymatic digestion of an antibody.

In certain embodiments, this method produces e.g. Fab or F(ab)2 antibody fragments. In certain embodiments, this method involves digestion with pepsin or papain, which are optionally immobilized on a surface.

In certain embodiments, antibodies may be humanized by CDR-grafting, in particular by a process involving the steps:
extracting RNA from hybridomas expressing an antibody of interest (e.g. obtained by a method as described herein);
amplifying said extracted RNA via RT-PCR, in particular with primer sets specific for the heavy and light chains of the antibody of interest, to obtain to obtain a DNA product;
further amplifying said DNA product via PCR, in particular using semi-nested primer sets specific for antibody variable regions;
determining the sequence of the DNA product;
aligning said sequence with homologous human framework sequences to determine a humanized sequence for the variable heavy chain and the variable light chain sequences (of the desired antibody).

In certain embodiments, antibodies may be humanized by aligning the sequence of a DNA product that was obtained by amplifying RNA extracted from hybridomas expressing an antibody of interest via RT-PCR, in particular with primer sets specific for the heavy and light chains of the antibody of interest and further amplifying the DNA obtained therefrom via PCR, in particular using semi-nested primer sets specific for antibody variable regions, with homologous human framework sequences to determine a humanized sequence for the variable heavy chain and the variable light chain sequences (of the desired antibody).

### In Certain Embodiments, Antibodies May be Humanized by

determining the complementary determining regions (CDR), which may be accomplished by analysing the structural interaction of framework regions (FR) with the complementary determining regions (CDR) and the antigen;
transplanting said CDR sequences into a human framework region.

In certain embodiments, antibodies may be humanized by transplanting CDR sequences, which may preferably have been determined by analysing the structural interaction of framework regions (FR) with the complementary determining regions (CDR) and the antigen, into a human framework region.

In certain embodiments variations in the amino acid sequence of the CDRs or FRs may be introduced to maintain structural interactions with the antigen (which may otherwise be abolished by introducing the human FR sequences), for instance by a random approach using phage display libraries or via directed approach guided by molecular modelling.

The DNA sequences encoding for antibodies determined as detailed herein can be transferred by known genetic engineering techniques into cells and used for production of the antibody.

### Producing Antibodies

In a further aspect, the antibody according to the present invention is a monoclonal antibody obtainable by the methods described herein, produced by a method comprising:
culturing a cell strain comprising a nucleotide sequence encoding for the antibody;
isolating the antibody from said culture.

In a further certain aspect, the antibody according to the present invention is a monoclonal antibody obtainable by the methods described herein, produced by isolating the antibody from a culture of a cell strain comprising a nucleotide sequence encoding for said antibody.

In certain embodiments of said method, the cell strain is produced as described herein above and may comprise bacterial cells, such as gram-negative bacteria, e.g. E. coli, Proteus mirabilis, or Pseudomonas putidas, gram-positive bacteria, e.g. Bacillus brevis, Bacillus subtilis, Bacillus megaterium, Lactobacilli such as Lactobacillus zeae/casei or Lactobacillus paracasei, or Streptomyces, such as Streptomyces lividans; eucaryotic cells such as yeast, e.g. Pichia pastoris, Saccharomyces cerevisiae, Hansenula polymorpha, Schizosaccharomyces pombe, Schwanniomyces occidentalis, Kluyveromyces lactis, or Yarrowia lipolytica; fugi, such as filamentous fungi, e.g. of the genus Trichoderma of Aspergillus, such as A. niger (e.g. subgenus A. awamori) and Aspergillus oryzae, Trichoderma reesei, Chrysosporium, such as C. lucknowense; protozoa, such as Leishmania, e.g. L. tarentolae; insect cells, such as insect cells transfected a Baculovirus, e.g. AcNPV, such as insect cell lines from Spodoptera frugiperda, e.g. Sf-9 or Sf-21, Drosophila melanogaster, e.g. DS2, or Trichoplusia ni, e.g. High Five cells (BTI-TN-5B1-4); mammalian cells such as hamster, e.g. Chinese hamster ovary such as K1-, DukX B11-, DG44, Lec13, or BHK, mouse, e.g. mouse myeloma such as NS0, Homo sapiens, e.g. Per.C6, AGE1LHN, HEK293.

In certain embodiments of said method, the cells may be hybridoma cells, e.g. as described herein.

In certain embodiments of said method, culturing may take place in a static suspension culture, an agitated suspension culture, a membrane-based culture, a matrix-based culture or a high cell density bioreactor; a vessel for such culturing may be selected from the group comprising a T-flask, a roller culture, a spinner culture, a stirred tank bioreactor, an airlift bioreactor, a static membrane-based or matrix-based culture system, a suspension bioreactor, a fluidized bed bioreactor, a ceramic bioreactor, a perfusion system, a hollow fiber bioreactor.

In certain embodiments of said method, the cells may be immobilized on a matrix.

A high cell density bioreactor is in particular a culture system capable of generating cell densities greater than 10⁸ cells/ml.

In a further aspect, the antibody according to the present invention is a monoclonal antibody obtainable by the methods described herein, produced by a method comprising:
generating a transgenic plant or animal comprising a nucleotide sequence encoding for the antibody;
isolating the antibody from said plant or animal or a secretion or product of said plant or animal.

In a certain further aspect, the antibody according to the present invention is a monoclonal antibody obtainable by the methods described herein, produced by isolating the antibody from a transgenic plant or transgenic animal or a secretion or product of a transgenic plant or transgenic animal having a nucleotide sequence encoding for the antibody.

Said animal may e.g., be selected from a chicken, a mouse, a rat, a rabbit, a cow, a goat, a sheep, a pig; said secretion or product may e.g. be milk or an egg. Said plant may e.g. be selected from tobacco (N. tabacum or N. benthamiana), duckweed (Lemna minor), Chlamydomonas reinhardtii, rice, Arabidopsis thaliana, alfalfa (Medicago sativa), lettuce, maize.

The antibodies can in certain embodiments be isolated by physicochemical fractionation, e.g. size exclusion chromatography, precipitation, e.g. using ammonium sulphate, ion exchange chromatography, immobilized metal chelate chromatography gel filtration, zone electrophoresis; based on their classification e.g. binding to bacterial proteins A, G, or L, jacalin; antigen-specific affinity purification via immobilized ligands/antigens; if necessary, low molecular weight components can be removed by methods like dialysis, desalting, and diafiltration.

In some embodiments the antibody is encoded by a nucleotide sequence where the nucleotide sequence is a reverse transcription of an amino acid sequence from an antibody produced by one of the processes described herein.

Humanization of murine antibodies may be conducted according to the following procedure:

For humanization of an antibody of murine origin the antibody sequence is analyzed for the structural interaction of framework regions (FR) with the complementary determining regions (CDR) and the antigen. Based on structural modelling an appropriate FR of human origin is selected and the murine CDR sequences are transplanted into the human FR. Variations in the amino acid sequence of the CDRs or FRs may be introduced to regain structural interactions, which were abolished by the species switch for the FR sequences. This recovery of structural interactions may be achieved by random approach using phage display libraries or via directed approach guided by molecular modelling (Almagro and Fransson 2008. Humanization of antibodies. Front Biosci. 2008 Jan. 1; 13:1619-33).

With the above context, the following consecutively numbered embodiments provide further specific aspects A of the invention:
1. Method for the prediction of a risk of side effects and/or prediction of the severity of side effects in a cancer patient that is to be treated or has been treated with immune effector cells in the frame of an immune effector cell therapy (IECT), and/or for assessing an effect of a treatment of side effects in a cancer patient that has been treated with immune effector cells in the frame of an immune effector cell therapy (IECT), the method comprising the steps of:
   determining the level of at least one endothelial dysfunction biomarker in a sample of bodily fluid of said patient, and
   correlating said level of said at least one endothelial dysfunction biomarker in said sample to the risk of said side effects and/or the severity of said side effects in said patient and/or to the effect of said treatment of said side effects in said patient.
2. The method according to item 1, wherein if said level of at least one endothelial dysfunction biomarker in a sample of bodily fluid obtained from said patient is above a threshold, said patient is at risk of developing said side effects and/or said treatment of side effects is being determined as being not effective.
3. The method according to items 1 and 2, wherein said side effects are selected from the group comprising cytokine release syndrome (CRS), and immune effector cell-associated neurotoxicity syndrome (ICANS).
4. The method according to item 3, wherein the severity of said cytokine release syndrome (CRS) or immune effector cell-associated neurotoxicity syndrome (ICANS) is mild (grade 1), moderate (grade 2), severe (3) or life-threatening (grade 4) or leads to death (grade 5).
5. The method according to items 1 to 4, wherein said immune effector cell therapy (IECT) is selected from the group comprising Chimeric Antigen Receptor T-cell (CAR-T) therapy, natural killer cell (NK) therapy, chimeric antigen receptor natural killer cell (CAR-NK) therapy, T cell receptor-engineered T-cell (TCR T) therapy, tumor-infiltrating T cell (TIT)), and cytokine-induced killer cell (CIK) therapy.
6. The method according to items 1 to 5, wherein said endothelial dysfunction biomarker is selected from the group comprising Proadrenomedullin (proADM) or fragments thereof, adhesion molecules (e.g. E-selectin, P-selectin, ICAM-1, VCAM-1), molecules involved in the coagulation pathway (e.g. von Willebrand factor (vWF) and soluble thrombomodulin), endoglin, endocan, IL-6, CRP, LOX-1, CD40L, ADMA, proendothelin-1 or fragments thereof (in particular ET-1, Big-ET, NT-proET-1), matrix metalloproteinases (MMPs, e.g. MMP-7 and MMP-9), and tissue inhibitor of metalloproteinases (TIMPs, e.g. TIMP-1), particularly selected from the group comprising proADM (SEQ ID NO. 2) or fragments thereof, adhesion molecules (e.g. E-selectin, P-selectin, ICAM-1, VCAM-1), molecules involved in the coagulation pathway (e.g. von Willebrand factor (vWF) and soluble thrombomodulin), endoglin, endocan, proendothelin-1 or fragments thereof (in particular ET-1, Big-ET, NT-proET-1), matrix metalloproteinases (MMPs, e.g. MMP-7 and MMP-9), and tissue inhibitor of metalloproteinases (TIMPs, e.g. TIMP-1).
7. The method according to item 6, wherein said fragment of Pro-Adrenomedullin is selected from the group comprising, PAMP-Gly (SEQ ID NO: 3), mature PAMP (SEQ ID NO: 4), MR-proADM (SEQ ID NO: 5), ADM-Gly (SEQ ID NO: 6), mature ADM (SEQ ID NO: 7) and CT-proADM (SEQ ID NO: 8);
   and wherein in particular the endothelial dysfunction biomarker is selected from the group comprising proADM and fragments thereof, which fragments are in particular PAMP-Gly, mature PAMP, MR-proADM, ADM-Gly, mature ADM and CT-proADM.
8. The method according to item 7, wherein said fragment of Pro-Adrenomedullin is MR-proADM and the threshold of the level of MR-proADM in a sample of bodily fluid of said patient is between 0.5 and 2 nmol/L, more preferred between 0.6 and 1.5 nmol/L, even more preferred between 0.7 and 1 nmol/L, most preferred said threshold is 0.8 nmol/L,
   or said threshold is an x-fold of the median level of MR-proADM in a healthy population, in particular in the range between 1.2-fold and 4.9-fold, more preferred between 1.5-fold and 3.7-fold, even more preferred between 1.7-fold and 2.4-fold, most preferred said threshold is 2.0-fold of the median of the level of MR-proADM in a healthy population.
9. The method according to item 7, wherein said fragment of Pro-Adrenomedullin is mature PAMP and the threshold of the level of mature PAMP in a sample of bodily fluid of said patient is between 0.7 and 1.2 pmol/L, more preferred between 0.8 and 1.0 pmol/L, most preferred said threshold is 0.9 pmol/L,
   or said threshold is an x-fold of the mean level of mature PAMP in a healthy population, in particular in the range between the 1.4-fold and 2.4-fold, more particular in the range between 1.6-fold and 2.0-fold, most particular said threshold is the 1.8-fold of the mean of the level of mature PAMP in a healthy population.
10. The method according to item 7, wherein said fragment of Pro-Adrenomedullin is PAMP-Gly and the threshold of the level of PAMP-Gly in a sample of bodily fluid of said patient is between 1.5 and 2.4 pmol/L, even more preferred between 1.8 and 2.2 pmol/L, most preferred said threshold is 2 pmol/L and 2.4 pmol/L, even more preferred between 1.8 and 2.2 pmol/L, most preferred said threshold is 2 pmol/L,
   or said threshold is an x-fold of the mean level of PAMP-Gly in a healthy population, in particular in the range between the 1.3-fold and 2.1-fold, more particular in the range between 1.6-fold and 1.9-fold, most particular said threshold is the 1.7-fold of the mean of the level of PAMP-Gly in a healthy population.
11. The method according to item 7, wherein said fragment of Pro-Adrenomedullin is ADM-Gly and the threshold of the level of ADM-Gly in a sample of bodily fluid of said patient is between 25 and 125 pg/ml, more preferred between 30 and 100 pg/ml, even more preferred between 35 and 75 pg/ml, most preferred said threshold is 40 pg/ml,
   or said threshold is an x-fold of the median level of ADM-Gly in a healthy population, in particular in the range between the 0.9-fold and 4.6-fold, more particular in the range between the 1.1-fold and 3.7-fold, more particular in the range between 1.3-fold and 2.8-fold, most particular said threshold is the 1.5-fold of the median of the level of ADM-Gly in a healthy population.
12. The method according to item 7, wherein said fragment of Pro-Adrenomedullin is mature ADM and the threshold of the level of mature ADM in a sample of bodily fluid of said patient is between 35 and 125 pg/ml, more preferred between 40 and 100 pg/ml, even more preferred between 50 and 90 pg/ml, most preferred said threshold is 70 pg/ml,
   or said threshold is an x-fold of the median level of mature ADM in a healthy population, in particular in the range between the 2.6-fold and 9.1-fold, more particular in the range between the 2.9-fold and 7.3-fold, more particular in the range between 3.6-fold and 6.6-fold, most particular said threshold is the 5.1-fold of the median of the level of mature ADM in a healthy population.
13. The method according to item 7, wherein said fragment of Pro-Adrenomedullin is CT-proADM and the threshold of the level of CT-proADM in a sample of bodily fluid of said patient is between 75 and 350 pmol/L, more preferred between 100 and 250 pmol/L, even more preferred between 125 and 200 pmol/L, most preferred said threshold is 150 pmol/L,
   or said threshold is an x-fold of the median level of CT-proADM in a healthy population, in particular in the range between the 1.0-fold and 4.5-fold, more particular in the range between the 1.3-fold and 3.2-fold, more particular in the range between 1.6-fold and 2.6-fold, most particular said threshold is the 1.9-fold of the median of the level of CT-proADM in a healthy population.
14. The method according to items 1 to 13, wherein said sample of bodily fluid is selected from the group comprising whole blood, plasma and serum.
15. The method according to items 1 to 14, wherein said determination of the level of at least one endothelial dysfunction biomarker and said correlation is performed more than once in said patient,
   wherein particularly said determination of the level of at least one endothelial dysfunction biomarker and said correlation is performed once before administration of immune effector cells to said patient and one or more times after administration of immune effector cells to said patient; or alternatively said determination of the level of at least one endothelial dysfunction biomarker and said correlation is performed at least twice after administration of immune effector cells to said patient.
16. The method according to items 1 to 15, wherein for said patient that is to be treated with immune effector cells in the frame of an immune effector cell therapy (IECT) said determination of the level of at least one endothelial dysfunction biomarker and said correlation is carried out in the time span between lymphodepletion and administration of the immune effector cells, more particularly within 14 days, or within 7 days, or within 2 days before said immune effector cells are administered to said patient, even more particularly within 48 hours, or within 24 hours, or within 12 hours, or within 6, or within 2 hours, or within 1 hour before said immune effector cells are administered to said patient.
17. The method according to items 1 to 16, wherein said patient that has been treated with immune effector cells has not developed side effects from said treatment at the time-point of taking said sample.
18. The method according to items 1 to 17, wherein for said patient that has been treated with immune effector cells said determination of the level of at least one endothelial dysfunction biomarker and said correlation is carried out within 1 week, or within 2 days after said immune effector cells have been administered to said patient, even more particularly within 48 hours, or within 24 hours, or within 12 hours, or within 6, or within 3 hours after said immune effector cells have been administered to said patient.
19. The method according to items 1 to 18, wherein said treatment of side effects is selected from the group comprising antihistamines, non-steroidal anti-inflammatory drugs (NSAIDs), immunosuppressive therapy, vasopressors, fluids and supportive oxygen supply.
20. The method according to items 1 to 19, wherein said treatment of side effects is administered if an increased risk of said side effects is predicted for said patient and/or said patient is in need of adjustment of said treatment of side effects if said treatment of side effects is being determined as not effective.
21. The method according to item 19, wherein said immunosuppressive therapy is selected from the group comprising anti-IL-6 antibody (e.g. siltuximab), anti-IL-6 receptor antibody (e.g. tocilizumab), anti-TNF-antibody (e.g. etanercept, infliximab), anti-IL-1-antibody (e.g. anakinra), and/or corticosteroids.

With the above context, the following consecutively numbered embodiments provide further specific aspects B of the invention:
1. Method for prevention or treatment of side effects of immune effector cell therapy (IECT) in a cancer patient that is to be treated or has been treated with immune effector cells in the frame of an immune effector cell therapy (IECT), wherein the treatment is immunosuppressive therapy, wherein said patient is selected by a method comprising the steps:
   determining at least one endothelial dysfunction biomarker in a sample of bodily fluid obtained from said patient and
   correlating said level of at least one endothelial dysfunction biomarker in said sample to said risk of side effects, and
wherein said side effects are selected from the group comprising cytokine release syndrome (CRS), and immune effector cell-associated neurotoxicity syndrome (ICANS).
2. The method according to aspect B item 1, wherein if said level of at least one endothelial dysfunction biomarker in a sample of bodily fluid obtained from said patient is above a threshold, said patient is at risk of developing said side effects and/or said treatment of side effects is being determined as being not effective.
3. The method according to aspect B item 1 or 2, wherein the severity of said cytokine release syndrome (CRS) or immune effector cell-associated neurotoxicity syndrome (ICANS) is mild (grade 1), moderate (grade 2), severe (3) or life-threatening (grade 4) or leads to death (grade 5).
4. The method according to aspect B items 1 to 3, wherein said immune effector cell therapy (IECT) is selected from the group comprising Chimeric Antigen Receptor T-cell (CAR-T) therapy, natural killer cell (NK) therapy, chimeric antigen receptor natural killer cell (CAR-NK) therapy, T cell receptor-engineered T-cell (TCR T) therapy, tumor-infiltrating T cell (TIT)), and cytokine-induced killer cell (CIK) therapy.
5. The method according to aspect B items 1 to 4, wherein said endothelial dysfunction biomarker is selected from the group comprising Proadrenomedullin (proADM) or fragments thereof, adhesion molecules (e.g. E-selectin, P-selectin, ICAM-1, VCAM-1), molecules involved in the coagulation pathway (e.g. von Willebrand factor (vWF) and soluble thrombomodulin), endoglin, endocan, IL-6, CRP, LOX-1, CD40L, ADMA, proendothelin-1 or fragments thereof (in particular ET-1, Big-ET, NT-proET-1), matrix metalloproteinases (MMPs, e.g. MMP-7 and MMP-9), and tissue inhibitor of metalloproteinases (TIMPs, e.g. TIMP-1),
   particularly selected from the group comprising proADM (SEQ ID NO. 2) or fragments thereof, adhesion molecules (e.g. E-selectin, P-selectin, ICAM-1, VCAM-1), molecules involved in the coagulation pathway (e.g. von Willebrand factor (vWF) and soluble thrombomodulin), endoglin, endocan, proendothelin-1 or fragments thereof (in particular ET-1, Big-ET, NT-proET-1), matrix metalloproteinases (MMPs, e.g. MMP-7 and MMP-9), and tissue inhibitor of metalloproteinases (TIMPs, e.g. TIMP-1).
6. The method according to aspect B item 5, wherein said fragment of Pro-Adrenomedullin is selected from the group comprising, PAMP-Gly (SEQ ID NO: 3), mature PAMP (SEQ ID NO: 4), MR-proADM (SEQ ID NO: 5), ADM-Gly (SEQ ID NO: 6), mature ADM (SEQ ID NO: 7) and CT-proADM (SEQ ID NO: 8);
   and wherein in particular the endothelial dysfunction biomarker is selected from the group comprising proADM and fragments thereof, which fragments are in particular PAMP-Gly, mature PAMP, MR-proADM, ADM-Gly, mature ADM and CT-proADM.
7. The method according to aspect B item 6, wherein said fragment of Pro-Adrenomedullin is MR-proADM and the threshold of the level of MR-proADM in a sample of bodily fluid of said patient is between 0.5 and 2 nmol/L, more preferred between 0.6 and 1.5 nmol/L, even more preferred between 0.7 and 1 nmol/L, most preferred said threshold is 0.8 nmol/L,
   or said threshold is an x-fold of the median level of MR-proADM in a healthy population, in particular in the range between 1.2-fold and 4.9-fold, more preferred between 1.5-fold and 3.7-fold, even more preferred between 1.7-fold and 2.4-fold, most preferred said threshold is 2.0-fold of the median of the level of MR-proADM in a healthy population.
8. The method according to aspect B item 7, wherein said fragment of Pro-Adrenomedullin is mature PAMP and the threshold of the level of mature PAMP in a sample of bodily fluid of said patient is between 0.7 and 1.2 pmol/L, more preferred between 0.8 and 1.0 pmol/L, most preferred said threshold is 0.9 pmol/L,
   or said threshold is an x-fold of the mean level of mature PAMP in a healthy population, in particular in the range between the 1.4-fold and 2.4-fold, more particular in the range between 1.6-fold and 2.0-fold, most particular said threshold is the 1.8-fold of the mean of the level of mature PAMP in a healthy population.
9. The method according to aspect B item 7, wherein said fragment of Pro-Adrenomedullin is PAMP-Gly and the threshold of the level of PAMP-Gly in a sample of bodily fluid of said patient is between 1.5 and 2.4 pmol/L, even more preferred between 1.8 and 2.2 pmol/L, most preferred said threshold is 2 pmol/L and 2.4 pmol/L, even more preferred between 1.8 and 2.2 pmol/L, most preferred said threshold is 2 pmol/L,
   or said threshold is an x-fold of the mean level of PAMP-Gly in a healthy population, in particular in the range between the 1.3-fold and 2.1-fold, more particular in the range between 1.6-fold and 1.9-fold, most particular said threshold is the 1.7-fold of the mean of the level of PAMP-Gly in a healthy population.
10. The method according to aspect B item 7, wherein said fragment of Pro-Adrenomedullin is ADM-Gly and the threshold of the level of ADM-Gly in a sample of bodily fluid of said patient is between 25 and 125 pg/ml, more preferred between 30 and 100 pg/ml, even more preferred between 35 and 75 pg/ml, most preferred said threshold is 40 pg/ml,
   or said threshold is an x-fold of the median level of ADM-Gly in a healthy population, in particular in the range between the 0.9-fold and 4.6-fold, more particular in the range between the 1.1-fold and 3.7-fold, more particular in the range between 1.3-fold and 2.8-fold, most particular said threshold is the 1.5-fold of the median of the level of ADM-Gly in a healthy population.
11. The method according to aspect B item 7, wherein said fragment of Pro-Adrenomedullin is mature ADM and the threshold of the level of mature ADM in a sample of bodily fluid of said patient is between 35 and 125 pg/ml, more preferred between 40 and 100 pg/ml, even more preferred between 50 and 90 pg/ml, most preferred said threshold is 70 pg/ml,
   or said threshold is an x-fold of the median level of mature ADM in a healthy population, in particular in the range between the 2.6-fold and 9.1-fold, more particular in the range between the 2.9-fold and 7.3-fold, more particular in the range between 3.6-fold and 6.6-fold, most particular said threshold is the 5.1-fold of the median of the level of mature ADM in a healthy population.
12. The method according to aspect B item 7, wherein said fragment of Pro-Adrenomedullin is CT-proADM and the threshold of the level of CT-proADM in a sample of bodily fluid of said patient is between 75 and 350 pmol/L, more preferred between 100 and 250 pmol/L, even more preferred between 125 and 200 pmol/L, most preferred said threshold is 150 pmol/L,
   or said threshold is an x-fold of the median level of CT-proADM in a healthy population, in particular in the range between the 1.0-fold and 4.5-fold, more particular in the range between the 1.3-fold and 3.2-fold, more particular in the range between 1.6-fold and 2.6-fold, most particular said threshold is the 1.9-fold of the median of the level of CT-proADM in a healthy population.
13. The method according to aspect B items 1 to 12, wherein said sample of bodily fluid is selected from the group comprising whole blood, plasma and serum.
14. The method according to aspect B items 1 to 13, wherein said determination of the level of at least one endothelial dysfunction biomarker and said correlation is performed more than once in said patient,
   wherein particularly said determination of the level of at least one endothelial dysfunction biomarker and said correlation is performed once before administration of immune effector cells to said patient and one or more times after administration of immune effector cells to said patient; or alternatively said determination of the level of at least one endothelial dysfunction biomarker and said correlation is performed at least twice after administration of immune effector cells to said patient.
15. The method according to aspect B items 1 to 14, wherein for said patient that is to be treated with immune effector cells in the frame of an immune effector cell therapy (IECT) said determination of the level of at least one endothelial dysfunction biomarker and said correlation is carried out in the time span between lymphodepletion and administration of the immune effector cells, more particularly within 14 days, or within 7 days, or within 2 days before said immune effector cells are administered to said patient, even more particularly within 48 hours, or within 24 hours, or within 12 hours, or within 6, or within 2 hours, or within 1 hour before said immune effector cells are administered to said patient.
16. The method according to aspect B items 1 to 15, wherein said patient that has been treated with immune effector cells has not developed side effects from said treatment at the time-point of taking said sample.
17. The method according to aspect B items 1 to 16, wherein for said patient that has been treated with immune effector cells said determination of the level of at least one endothelial dysfunction biomarker and said correlation is carried out within 1 week, or within 2 days after said immune effector cells have been administered to said patient, even more particularly within 48 hours, or within 24 hours, or within 12 hours, or within 6, or within 3 hours after said immune effector cells have been administered to said patient.
18. The method according to aspect B items 1 to 17, wherein said treatment of side effects is selected from the group comprising antihistamines, non-steroidal anti-inflammatory drugs (NSAIDs), immunosuppressive therapy, vasopressors, fluids and supportive oxygen supply.
19. The method according to aspect B items 1 to 18, wherein said treatment of side effects is administered if an increased risk of said side effects is predicted for said patient and/or said patient is in need of adjustment of said treatment of side effects if said treatment of side effects is being determined as not effective.
20. The method according to aspect B item 19, wherein said immunosuppressive therapy is selected from the group comprising anti-IL-6 antibody (e.g. siltuximab), anti-IL-6 receptor antibody (e.g. tocilizumab), anti-TNF-antibody (e.g. etanercept, infliximab), anti-IL-1-antibody (e.g. anakinra), and/or corticosteroids.

With the above context, the following consecutively numbered embodiments provide further specific aspects C of the invention:
1. Medicament for use in the prevention or treatment of side effects of immune effector cell therapy (IECT) in a cancer patient that is to be treated or has been treated with immune effector cells in the frame of an immune effector cell therapy (IECT), wherein said patient is selected by a method comprising the steps:
   determining at least one endothelial dysfunction biomarker in a sample of bodily fluid obtained from said patient and
   correlating said level of at least one endothelial dysfunction biomarker in said sample to said risk of side effects, and
wherein said side effects are selected from the group comprising cytokine release syndrome (CRS), and immune effector cell-associated neurotoxicity syndrome (ICANS), and wherein said medicament is an immunosuppressive medicament.
2. The medicament for use according to aspect C item 1, wherein if said level of at least one endothelial dysfunction biomarker in a sample of bodily fluid obtained from said patient is above a threshold, said patient is at risk of developing said side effects and said medicament is to be administered to said patient.
3. The medicament for use according to aspect C item 1 or 2, wherein the severity of said cytokine release syndrome (CRS) or immune effector cell-associated neurotoxicity syndrome (ICANS) is mild (grade 1), moderate (grade 2), severe (3) or life-threatening (grade 4) or leads to death (grade 5).
4. The medicament for use according to aspect C items 1 to 3, wherein said immune effector cell therapy (IECT) is selected from the group comprising Chimeric Antigen Receptor T-cell (CAR-T) therapy, natural killer cell (NK) therapy, chimeric antigen receptor natural killer cell (CAR-NK) therapy, T cell receptor-engineered T-cell (TCR T) therapy, tumor-infiltrating T cell (TIT)), and cytokine-induced killer cell (CIK) therapy.
5. The medicament for use according to aspect C items 1 to 4, wherein said endothelial dysfunction biomarker is selected from the group comprising Proadrenomedullin (proADM) or fragments thereof, adhesion molecules (e.g. E-selectin, P-selectin, ICAM-1, VCAM-1), molecules involved in the coagulation pathway (e.g. von Willebrand factor (vWF) and soluble thrombomodulin), endoglin, endocan, IL-6, CRP, LOX-1, CD40L, ADMA, proendothelin-1 or fragments thereof (in particular ET-1, Big-ET, NT-proET-1), matrix metalloproteinases (MMPs, e.g. MMP-7 and MMP-9), and tissue inhibitor of metalloproteinases (TIMPs, e.g. TIMP-1),
   particularly selected from the group comprising proADM (SEQ ID NO. 2) or fragments thereof, adhesion molecules (e.g. E-selectin, P-selectin, ICAM-1, VCAM-1), molecules involved in the coagulation pathway (e.g. von Willebrand factor (vWF) and soluble thrombomodulin), endoglin, endocan, proendothelin-1 or fragments thereof (in particular ET-1, Big-ET, NT-proET-1), matrix metalloproteinases (MMPs, e.g. MMP-7 and MMP-9), and tissue inhibitor of metalloproteinases (TIMPs, e.g. TIMP-1).
6. The medicament for use according to aspect C item 5, wherein said fragment of Pro-Adrenomedullin is selected from the group comprising, PAMP-Gly (SEQ ID NO: 3), mature PAMP (SEQ ID NO: 4), MR-proADM (SEQ ID NO: 5), ADM-Gly (SEQ ID NO: 6), mature ADM (SEQ ID NO: 7) and CT-proADM (SEQ ID NO: 8);
   and wherein in particular the endothelial dysfunction biomarker is selected from the group comprising proADM and fragments thereof, which fragments are in particular PAMP-Gly, mature PAMP, MR-proADM, ADM-Gly, mature ADM and CT-proADM.
7. The medicament for use according to aspect C item 7, wherein said fragment of Pro-Adrenomedullin is MR-proADM and the threshold of the level of MR-proADM in a sample of bodily fluid of said patient is between 0.5 and 2 nmol/L, more preferred between 0.6 and 1.5 nmol/L, even more preferred between 0.7 and 1 nmol/L, most preferred said threshold is 0.8 nmol/L,
   or said threshold is an x-fold of the median level of MR-proADM in a healthy population, in particular in the range between 1.2-fold and 4.9-fold, more preferred between 1.5-fold and 3.7-fold, even more preferred between 1.7-fold and 2.4-fold, most preferred said threshold is 2.0-fold of the median of the level of MR-proADM in a healthy population.
8. The medicament for use according to aspect C item 7, wherein said fragment of Pro-Adrenomedullin is mature PAMP and the threshold of the level of mature PAMP in a sample of bodily fluid of said patient is between 0.7 and 1.2 pmol/L, more preferred between 0.8 and 1.0 pmol/L, most preferred said threshold is 0.9 pmol/L,
   or said threshold is an x-fold of the mean level of mature PAMP in a healthy population, in particular in the range between the 1.4-fold and 2.4-fold, more particular in the range between 1.6-fold and 2.0-fold, most particular said threshold is the 1.8-fold of the mean of the level of mature PAMP in a healthy population.
9. The medicament for use according to aspect C item 7, wherein said fragment of Pro-Adrenomedullin is PAMP-Gly and the threshold of the level of PAMP-Gly in a sample of bodily fluid of said patient is between 1.5 and 2.4 pmol/L, even more preferred between 1.8 and 2.2 pmol/L, most preferred said threshold is 2 pmol/L and 2.4 pmol/L, even more preferred between 1.8 and 2.2 pmol/L, most preferred said threshold is 2 pmol/L,
   or said threshold is an x-fold of the mean level of PAMP-Gly in a healthy population, in particular in the range between the 1.3-fold and 2.1-fold, more particular in the range between 1.6-fold and 1.9-fold, most particular said threshold is the 1.7-fold of the mean of the level of PAMP-Gly in a healthy population.
10. The medicament for use according to aspect C item 7, wherein said fragment of Pro-Adrenomedullin is ADM-Gly and the threshold of the level of ADM-Gly in a sample of bodily fluid of said patient is between 25 and 125 pg/ml, more preferred between 30 and 100 pg/ml, even more preferred between 35 and 75 pg/ml, most preferred said threshold is 40 pg/ml,
   or said threshold is an x-fold of the median level of ADM-Gly in a healthy population, in particular in the range between the 0.9-fold and 4.6-fold, more particular in the range between the 1.1-fold and 3.7-fold, more particular in the range between 1.3-fold and 2.8-fold, most particular said threshold is the 1.5-fold of the median of the level of ADM-Gly in a healthy population.
11. The medicament for use according to aspect C item 7, wherein said fragment of Pro-Adrenomedullin is mature ADM and the threshold of the level of mature ADM in a sample of bodily fluid of said patient is between 35 and 125 pg/ml, more preferred between 40 and 100 pg/ml, even more preferred between 50 and 90 pg/ml, most preferred said threshold is 70 pg/ml, or said threshold is an x-fold of the median level of mature ADM in a healthy population, in particular in the range between the 2.6-fold and 9.1-fold, more particular in the range between the 2.9-fold and 7.3-fold, more particular in the range between 3.6-fold and 6.6-fold, most particular said threshold is the 5.1-fold of the median of the level of mature ADM in a healthy population.
12. The medicament for use according to aspect C item 7, wherein said fragment of Pro-Adrenomedullin is CT-proADM and the threshold of the level of CT-proADM in a sample of bodily fluid of said patient is between 75 and 350 pmol/L, more preferred between 100 and 250 pmol/L, even more preferred between 125 and 200 pmol/L, most preferred said threshold is 150 pmol/L,
   or said threshold is an x-fold of the median level of CT-proADM in a healthy population, in particular in the range between the 1.0-fold and 4.5-fold, more particular in the range between the 1.3-fold and 3.2-fold, more particular in the range between 1.6-fold and 2.6-fold, most particular said threshold is the 1.9-fold of the median of the level of CT-proADM in a healthy population.
13. The medicament for use according to aspect C items 1 to 12, wherein said sample of bodily fluid is selected from the group comprising whole blood, plasma and serum.
14. The medicament for use according to aspect C items 1 to 13, wherein said determination of the level of at least one endothelial dysfunction biomarker and said correlation is performed more than once in said patient,
   wherein particularly said determination of the level of at least one endothelial dysfunction biomarker and said correlation is performed once before administration of immune effector cells to said patient and one or more times after administration of immune effector cells to said patient; or alternatively said determination of the level of at least one endothelial dysfunction biomarker and said correlation is performed at least twice after administration of immune effector cells to said patient.
15. The medicament for use according to aspect C items 1 to 14, wherein for said patient that is to be treated with immune effector cells in the frame of an immune effector cell therapy (IECT) said determination of the level of at least one endothelial dysfunction biomarker and said correlation is carried out in the time span between lymphodepletion and administration of the immune effector cells, more particularly within 14 days, or within 7 days, or within 2 days before said immune effector cells are administered to said patient, even more particularly within 48 hours, or within 24 hours, or within 12 hours, or within 6, or within 2 hours, or within 1 hour before said immune effector cells are administered to said patient.
16. The medicament for use according to aspect C items 1 to 15, wherein said patient that has been treated with immune effector cells has not developed side effects from said treatment at the time-point of taking said sample.
17. The medicament for use according to aspect C items 1 to 16, wherein for said patient that has been treated with immune effector cells said determination of the level of at least one endothelial dysfunction biomarker and said correlation is carried out within 1 week, or within 2 days after said immune effector cells have been administered to said patient, even more particularly within 48 hours, or within 24 hours, or within 12 hours, or within 6, or within 3 hours after said immune effector cells have been administered to said patient.
18. The medicament for use according to aspect C item 1 to 17, wherein said immunosuppressive therapy is selected from the group comprising anti-IL-6 antibody (e.g. siltuximab), anti-IL-6 receptor antibody (e.g. tocilizumab), anti-TNF-antibody (e.g. etanercept, infliximab), anti-IL-1-antibody (e.g. anakinra), and/or corticosteroids.

With the above context, the following consecutively numbered embodiments provide further specific aspects D of the invention:
1. A method for patient stratification and/or selection of a patient for treatment of side effects, wherein said patient is a cancer patient that is to be treated or has been treated with immune effector cells in the frame of an immune effector cell therapy (IECT), said method comprising:
   determining the level of at least one endothelial dysfunction biomarker in a sample of bodily fluid of said patient and
   assigning if said patient is to receive treatment of said side effects based on said level of at least one endothelial dysfunction biomarker in said sample.
2. The method according to aspect D item 1, wherein if said level of at least one endothelial dysfunction biomarker in a sample of bodily fluid obtained from said patient is above a threshold, said patient is assigned to receive treatment of said side effects.
3. The method according to aspect D items 1 and 2, wherein said side effects are selected from the group comprising cytokine release syndrome (CRS), and immune effector cell-associated neurotoxicity syndrome (ICANS).
4. The method according to aspect D item 3, wherein the severity of said cytokine release syndrome (CRS) or immune effector cell-associated neurotoxicity syndrome (ICANS) is mild (grade 1), moderate (grade 2), severe (3) or life-threatening (grade 4) or leads to death (grade 5).
5. The method according to aspect D items 1 to 4, wherein said immune effector cell therapy (IECT) is selected from the group comprising Chimeric Antigen Receptor T-cell (CAR-T) therapy, natural killer cell (NK) therapy, chimeric antigen receptor natural killer cell (CAR-NK) therapy, T cell receptor-engineered T-cell (TCR T) therapy, tumor-infiltrating T cell (TIT)), and cytokine-induced killer cell (CIK) therapy.
6. The method according to aspect D items 1 to 5, wherein said endothelial dysfunction biomarker is selected from the group comprising Proadrenomedullin (proADM) or fragments thereof, adhesion molecules (e.g. E-selectin, P-selectin, ICAM-1, VCAM-1), molecules involved in the coagulation pathway (e.g. von Willebrand factor (vWF) and soluble thrombomodulin), endoglin, endocan, IL-6, CRP, LOX-1, CD40L, ADMA, proendothelin-1 or fragments thereof (in particular ET-1, Big-ET, NT-proET-1), matrix metalloproteinases (MMPs, e.g. MMP-7 and MMP-9), and tissue inhibitor of metalloproteinases (TIMPs, e.g. TIMP-1), particularly selected from the group comprising proADM (SEQ ID NO. 2) or fragments thereof, adhesion molecules (e.g. E-selectin, P-selectin, ICAM-1, VCAM-1), molecules involved in the coagulation pathway (e.g. von Willebrand factor (vWF) and soluble thrombomodulin), endoglin, endocan, proendothelin-1 or fragments thereof (in particular ET-1, Big-ET, NT-proET-1), matrix metalloproteinases (MMPs, e.g. MMP-7 and MMP-9), and tissue inhibitor of metalloproteinases (TIMPs, e.g. TIMP-1).
7. The method according to aspect D item 6, wherein said fragment of Pro-Adrenomedullin is selected from the group comprising, PAMP-Gly (SEQ ID NO: 3), mature PAMP (SEQ ID NO: 4), MR-proADM (SEQ ID NO: 5), ADM-Gly (SEQ ID NO: 6), mature ADM (SEQ ID NO: 7) and CT-proADM (SEQ ID NO: 8);
   and wherein in particular the endothelial dysfunction biomarker is selected from the group comprising proADM and fragments thereof, which fragments are in particular PAMP-Gly, mature PAMP, MR-proADM, ADM-Gly, mature ADM and CT-proADM.
8. The method according to aspect D item 7, wherein said fragment of Pro-Adrenomedullin is MR-proADM and the threshold of the level of MR-proADM in a sample of bodily fluid of said patient is between 0.5 and 2 nmol/L, more preferred between 0.6 and 1.5 nmol/L, even more preferred between 0.7 and 1 nmol/L, most preferred said threshold is 0.8 nmol/L,
   or said threshold is an x-fold of the median level of MR-proADM in a healthy population, in particular in the range between 1.2-fold and 4.9-fold, more preferred between 1.5-fold and 3.7-fold, even more preferred between 1.7-fold and 2.4-fold, most preferred said threshold is 2.0-fold of the median of the level of MR-proADM in a healthy population.
9. The method according to aspect D item 7, wherein said fragment of Pro-Adrenomedullin is mature PAMP and the threshold of the level of mature PAMP in a sample of bodily fluid of said patient is between 0.7 and 1.2 pmol/L, more preferred between 0.8 and 1.0 pmol/L, most preferred said threshold is 0.9 pmol/L,
   or said threshold is an x-fold of the mean level of mature PAMP in a healthy population, in particular in the range between the 1.4-fold and 2.4-fold, more particular in the range between 1.6-fold and 2.0-fold, most particular said threshold is the 1.8-fold of the mean of the level of mature PAMP in a healthy population.
10. The method according to aspect D item 7, wherein said fragment of Pro-Adrenomedullin is PAMP-Gly and the threshold of the level of PAMP-Gly in a sample of bodily fluid of said patient is between 1.5 and 2.4 pmol/L, even more preferred between 1.8 and 2.2 pmol/L, most preferred said threshold is 2 pmol/L and 2.4 pmol/L, even more preferred between 1.8 and 2.2 pmol/L, most preferred said threshold is 2 pmol/L,
   or said threshold is an x-fold of the mean level of PAMP-Gly in a healthy population, in particular in the range between the 1.3-fold and 2.1-fold, more particular in the range between 1.6-fold and 1.9-fold, most particular said threshold is the 1.7-fold of the mean of the level of PAMP-Gly in a healthy population.
11. The method according to aspect D item 7, wherein said fragment of Pro-Adrenomedullin is ADM-Gly and the threshold of the level of ADM-Gly in a sample of bodily fluid of said patient is between 25 and 125 pg/ml, more preferred between 30 and 100 pg/ml, even more preferred between 35 and 75 pg/ml, most preferred said threshold is 40 pg/ml,
   or said threshold is an x-fold of the median level of ADM-Gly in a healthy population, in particular in the range between the 0.9-fold and 4.6-fold, more particular in the range between the 1.1-fold and 3.7-fold, more particular in the range between 1.3-fold and 2.8-fold, most particular said threshold is the 1.5-fold of the median of the level of ADM-Gly in a healthy population.
12. The method according to aspect D item 7, wherein said fragment of Pro-Adrenomedullin is mature ADM and the threshold of the level of mature ADM in a sample of bodily fluid of said patient is between 35 and 125 pg/ml, more preferred between 40 and 100 pg/ml, even more preferred between 50 and 90 pg/ml, most preferred said threshold is 70 pg/ml,
   or said threshold is an x-fold of the median level of mature ADM in a healthy population, in particular in the range between the 2.6-fold and 9.1-fold, more particular in the range between the 2.9-fold and 7.3-fold, more particular in the range between 3.6-fold and 6.6-fold, most particular said threshold is the 5.1-fold of the median of the level of mature ADM in a healthy population.
13. The method according to aspect D item 7, wherein said fragment of Pro-Adrenomedullin is CT-proADM and the threshold of the level of CT-proADM in a sample of bodily fluid of said patient is between 75 and 350 pmol/L, more preferred between 100 and 250 pmol/L, even more preferred between 125 and 200 pmol/L, most preferred said threshold is 150 pmol/L,
   or said threshold is an x-fold of the median level of CT-proADM in a healthy population, in particular in the range between the 1.0-fold and 4.5-fold, more particular in the range between the 1.3-fold and 3.2-fold, more particular in the range between 1.6-fold and 2.6-fold, most particular said threshold is the 1.9-fold of the median of the level of CT-proADM in a healthy population.
14. The method according to aspect D items 1 to 13, wherein said sample of bodily fluid is selected from the group comprising whole blood, plasma and serum.
15. The method according to aspect D items 1 to 14, wherein said determination of the level of at least one endothelial dysfunction biomarker and said correlation is performed more than once in said patient,
   wherein particularly said determination of the level of at least one endothelial dysfunction biomarker and said correlation is performed once before administration of immune effector cells to said patient and one or more times after administration of immune effector cells to said patient; or alternatively said determination of the level of at least one endothelial dysfunction biomarker and said correlation is performed at least twice after administration of immune effector cells to said patient.
16. The method according to aspect D items 1 to 15, wherein for said patient that is to be treated with immune effector cells in the frame of an immune effector cell therapy (IECT) said determination of the level of at least one endothelial dysfunction biomarker and said correlation is carried out in the time span between lymphodepletion and administration of the immune effector cells, more particularly within 14 days, or within 7 days, or within 2 days before said immune effector cells are administered to said patient, even more particularly within 48 hours, or within 24 hours, or within 12 hours, or within 6, or within 2 hours, or within 1 hour before said immune effector cells are administered to said patient.
17. The method according to aspect D items 1 to 16, wherein said patient that has been treated with immune effector cells has not developed side effects from said treatment at the time-point of taking said sample.
18. The method according to aspect D items 1 to 17, wherein for said patient that has been treated with immune effector cells said determination of the level of at least one endothelial dysfunction biomarker and said correlation is carried out within 1 week, or within 2 days after said immune effector cells have been administered to said patient, even more particularly within 48 hours, or within 24 hours, or within 12 hours, or within 6, or within 3 hours after said immune effector cells have been administered to said patient.
19. The method according to aspect D items 1 to 18, wherein said treatment of side effects is selected from the group comprising antihistamines, non-steroidal anti-inflammatory drugs (NSAIDs), immunosuppressive therapy, vasopressors, fluids and supportive oxygen supply.
20. The method according to aspect D item 19, wherein said immunosuppressive therapy is selected from the group comprising anti-IL-6 antibody (e.g. siltuximab), anti-IL-6 receptor antibody (e.g. tocilizumab), anti-TNF-antibody (e.g. etanercept, infliximab), anti-IL-1-antibody (e.g. anakinra), and/or corticosteroids.

### FIGURE DESCRIPTION

FIG. 1: Representative calibration curve of the bio-ADM assay (in the presence or absence of 100 μg/mL antibody NT-H).

FIG. 2: Representative calibration curve of the ADM-Gly Assay with synthetic human 1-53 ADM-Gly

FIG. 3: Frequency distribution (histogram) of ADM-Gly (pg/mL) in self-reported healthy individuals (n=128)

FIG. 4: Concentrations of bio-ADM—(Panel A) and ADM-Gly (Panel B) in cancer patients prior to start of T-cell administration in the frame of a chimeric antigen receptor T-cell therapy (CAR T) dependent on the Cytokine Release Syndrome (CRS) Grading <2 or ≥2 following the CAR T-cell therapy. Shown are box and whiskers plots (with median, 25^{th} and 75^{th} percentiles and min and max data indicated).

FIG. 5: Time course of bio-ADM (Panel A) and ADM-Gly (Panel B) after CAR T-cell administration in the frame of CAR T-cell therapy dependent on the Cytokine Release Syndrome (CRS) Grading <2 or ≥2 following the CAR T-cell therapy. As blood draws were not taken at the exact same time points after start of CAR T-cell therapy for all patients, bio-ADM- and ADM-Gly measurements were grouped in time intervals of four days, e.g. >0 and ≤4 days, >4 and ≤8 days, >8 and ≤12 days, >12 and ≤16 days, >16 days, after start of CAR-T cell therapy. Displayed are bars for median with interquartile range.

### REFERENCES

Ayala Ceja, M., Khericha, M., Harris, C. M., Puig-Saus, C., & Chen, Y. Y. (2024). CAR-T cell manufacturing: major process parameters and next-generation strategies. Journal of Experimental Medicine, 221(2), e20230903.
Bilint, L., Nelson-Maney, N. P., Tian, Y., Serafin, S. D., & Caron, K. M. (2023). Clinical Potential of Adrenomedullin Signaling in the Cardiovascular System. Circulation Research, 132(9), 1185-1202. https://doi.org/10.1161/CIRCRESAHA.123.321673.
Chistiakov, D. A., Orekhov, A. N., & Bobryshev, Y. V. (2015). Endothelial barrier and its abnormalities in cardiovascular disease. Frontiers in Physiology, 6, 158540.
Chohan, K. L., Siegler, E. L., & Kenderian, S. S. (2023). CAR-T cell therapy: the efficacy and toxicity balance. Current Hematologic Malignancy Reports, 18(2), 9-18.
Claesson-Welsh, L. (2015). Vascular permeability—the essentials. Upsala Journal ofMedical Sciences, 120(3), 135-143.
Claesson-Welsh, L., Dejana, E., & McDonald, D. M. (2021). Permeability of the endothelial barrier: identifying and reconciling controversies. Trends in Molecular Medicine, 27(4), 314-331.
Eto, T. (2001). A review of the biological properties and clinical implications of adrenomedullin and Proadrenomedullin N-terminal 20 peptide (PAMP), hypotensive and vasodilating peptides.
Peptides, 22(11), 1693-1711. https://doi.org/https://doi.org/10.1016/S0196-9781(01)00513-7
Garcia, M. A., Martin-Santamaria, S., de Pascual-Teresa, B., Ramos, A., Juliin, M., & Martinez, A. (2006). Adrenomedullin: a new and promising target for drug discovery. Expert Opinion on Therapeutic Targets, 10(2), 303-317.
Gavriilaki, E., Sakellari, I., Gavriilaki, M., & Anagnostopoulos, A. (2020). A new era in endothelial injury syndromes: toxicity of CAR-T cells and the role of immunity. International Journal of Molecular Sciences, 21(11), 3886.
GGjer, E., Balat, A., Qekmen, M., Kiling, M., Sivasli, E., Yurekli, M., & Duman, C. (2006). The effects of anticancer drugs on levels of nitric oxide and adrenomedullin. The Turkish Journal of Pediatrics, 48(3), 202-208.
Jaime Garcia, D., Chagnot, A., Wardlaw, J. M., & Montagne, A. (2023). A scoping review on biomarkers of endothelial dysfunction in small vessel disease: molecular insights from human studies. International Journal of Molecular Sciences, 24(17), 13114.
Lee, D. W., Santomasso, B. D., Locke, F. L., Ghobadi, A., Turtle, C. J., Brudno, J. N., Maus, M. V, Park, J. H., Mead, E., & Pavletic, S. (2019). ASTCT consensus grading for cytokine release syndrome and neurologic toxicity associated with immune effector cells. Biology of Blood and Marrow Transplantation, 25(4), 625-638.
Lee, W. L., & Slutsky, A. S. (2010). Sepsis and endothelial permeability. New England Journal of Medicine, 363(7), 689-691.
Leite, A. R., Borges-Canha, M., Cardoso, R., Neves, J. S., Castro-Ferreira, R., & Leite-Moreira, A. (2020). Novel biomarkers for evaluation of endothelial dysfunction. Angiology, 71(5), 397-410.
Link, W. (2019). Cancer Therapy BT—Principles of Cancer Treatment and Anticancer Drug Development (W. Link (ed.); pp. 7-76). Springer International Publishing. https://doi.org/10.1007/978-3-030-18722-4_2
Neelapu, S. S., Tummala, S., Kebriaei, P., Wierda, W., Gutierrez, C., Locke, F. L., Komanduri, K. V, Lin, Y., Jain, N., & Daver, N. (2018). Chimeric antigen receptor T-cell therapy-assessment and management of toxicities. Nature Reviews Clinical Oncology, 15(1), 47-62.
O Karpinich, N., L Hoopes, S., O Kechele, D., M Lenhart, P., & M Caron, K. (2011). Adrenomedullin function in vascular endothelial cells: insights from genetic mouse models. Current Hypertension Reviews, 7(4), 228-239.
Opal, S. M., & Van Der Poll, T. (2015). Endothelial barrier dysfunction in septic shock. Journal of Internal Medicine, 277(3), 277-293.
Rodrigues, S. F., & Granger, D. N. (2015). Blood cells and endothelial barrier function. Tissue Barriers, 3(1-2), e978720.
Rosen, R. S., Yang, J. H., Pena, J. S., Schloss, R., & Yarmush, M. L. (2023). An in vitro model of the macrophage-endothelial interface to characterize CAR T-cell induced cytokine storm. Scientific Reports, 13(1), 18835.
Soussi, S., dos Santos, C., Jentzer, J. C., Mebazaa, A., Gayat, E., Pass, J., Schaubroeck, H., Billia, F., Marshall, J. C., & Lawler, P. R. (2023). Distinct host-response signatures in circulatory shock: a narrative review. Intensive Care Medicine Experimental, 11(1). https://doi.org/10.1186/s40635-023-00531-5
Sumransub, N., El Jurdi, N., Chiraphapphaiboon, W., & Maakaron, J. E. (2022). Putting function back in dysfunction: Endothelial diseases and current therapies in hematopoietic stem cell transplantation and cellular therapies. Blood Reviews, 51, 100883.
Sun, D., Shi, X., Li, S., Wang, X., Yang, X., & Wan, M. (2024). CAR-T cell therapy: A breakthrough in traditional cancer treatment strategies. Molecular Medicine Reports, 29(3), 1-9.
van Lier, D., Kox, M., & Pickkers, P. (2020). Promotion of vascular integrity in sepsis through modulation of bioactive adrenomedullin and dipeptidyl peptidase 3. Journal of Internal Medicine, 1-15. https://doi.org/10.1111/joim.13220 Zhang, J. (2022). Biomarkers of endothelial activation and dysfunction in cardiovascular diseases.
Reviews in Cardiovascular Medicine, 23(2), 73.

### EXAMPLES

### Example 1—Generation of Anti-ADM Antibodies and Determination of their Affinity Constants

We developed mouse monoclonal antibodies binding to the N-terminal (NT-ADM), mid-regional (MR-ADM) and C-terminal (CT-ADM) part of bio-ADM and their affinity constants were determined (Table 1). Peptides were supplied by JPT Peptide Technologies GmbH (Berlin, Germany). Peptides were coupled to BSA using the Sulfo-SMCC crosslinking method. The crosslinking procedure was performed according the manufacturer's instructions (Thermo Fisher/Pierce).

### Generation of Murine Antibodies

A Balb/c mouse was immunized with 100 μg Peptide-BSA-Conjugate at day 0 and 14 (emulsified in 100 μl complete Freund's adjuvant) and 50 μg at day 21 and 28 (in 100 μl incomplete Freund's adjuvant). Three days before the fusion experiment was performed, the animal received 50 μg of the conjugate dissolved in 100 μl saline, given as one intraperitoneal and one intra venous injection.

Splenocytes from the immunized mouse and cells of the myeloma cell line SP2/0 were fused with 1 ml 50% polyethylene glycol for 30 s at 37° C. After washing, the cells were seeded in 96-well cell culture plates. Hybrid clones were selected by growing in HAT medium (RPMI 1640 culture medium supplemented with 20% fetal calf serum and HAT-Supplement). After two weeks the HAT medium is replaced with HT medium for three passages followed by returning to the normal cell culture medium. The cell culture supernatants were primary screened for antigen specific IgG antibodies three weeks after fusion. The positive tested microcultures were transferred into 24-well plates for propagation. After retesting the selected cultures were cloned and recloned using the limiting-dilution technique and the isotypes were determined (Lane, 1985. J. Immunol. Meth. 81: 223-228; Ziegler et al. 1996. Horm. Metab. Res. 28: 11-15).

TABLE 1
ADM-Peptides for immunization and anti-ADM antibodies

| | ADM | | Affinity constants |
|---|---|---|---|
| Antigen/Immunogen | Region | Designation | Kd (M⁻¹) |
| YRQSMNNFQGLRSFGC (SEQ ID NO: 9) | 1-16 | NT-ADM | 1.6 × 10⁹ |
| CTVQKLAHQIYQ (SEQ ID NO: 10) | 21-32 | MR-ADM | 2 × 10⁹ |
| CAPRSKISPQGY-NH₂ (SEQ ID NO: 11) | C-42-52 | CT-ADM | 1.1 × 10⁹ |

### Monoclonal Antibody Production

Antibodies were produced via standard antibody production methods (Marx et al, 1997. Monoclonal Antibody Production, ATLA 25, 121) and purified via Protein A. The antibody purities were >95% based on SDS gel electrophoresis analysis.

### Affinity Constants

To determine the affinity of the antibodies to Adrenomedullin, the kinetics of binding of Adrenomedullin to immobilized antibody was determined by means of label-free surface plasmon resonance using a Biacore 2000 system (GE Healthcare Europe GmbH, Freiburg, Germany). Reversible immobilization of the antibodies was performed using an anti-mouse Fe antibody covalently coupled in high density to a CM5 sensor surface according to the manufacturer's instructions (mouse antibody capture kit; GE Healthcare).

### Labelling Procedure (Tracer)

100 μg (100 μl) of antibody (1 mg/ml in PBS, pH 7.4) was mixed with 10 μl Akridinium NHS-ester (1 mg/ml in acetonitrile, InVent GmbH, Germany) (EP 0 353 971) and incubated for 20 min at room temperature. Labelled CT-H was purified by Gel-filtration HPLC on Bio-Sil® SEC 400-5 (Bio-Rad Laboratories, Inc., USA). The purified labeled antibody was diluted in (300 mmol/L potassium phosphate, 100 mmol/L NaCl, 10 mmol/L Na-EDTA, 5 g/L Bovine Serum Albumin, pH 7.0). The final concentration was approx. 800.000 relative light units (RLU) of labelled compound (approx. 20 ng labeled antibody) per 200 μL. Akridiniumester chemiluminescence was measured by using an AutoLumat LB 953 (Berthold Technologies GmbH & Co. KG).

### Solid Phase

Polystyrene tubes (Greiner Bio-One International AG, Austria) were coated (18 h at room temperature) with antibody (1.5 μg antibody/0.3 mL 100 mmol/L NaCl, 50 mmol/L TRIS/HCl, pH 7.8). After blocking with 5% bovine serum albumin, the tubes were washed with PBS, pH 7.4 and vacuum dried.

### Calibrators

Synthetic human ADM (hADM) (Bachem, Switzerland) was linearly diluted using 50 mM Tris/HCl, 250 mM NaCl, 0.2% Triton X-100, 0.5% BSA, 20 tabs/L Protease Complete Protease Inhibitor Cocktail Tablets (Roche AG); pH 7.8. Calibrators were stored at −20° C. before use.

### Example 2—Determination of the Antibody Combination that Yields High Signal/Noise Ratios ADM Immunoassay

50 μl of sample (or calibrator) was pipetted into coated tubes, after adding labelled second antibody (200 μl), the tubes were incubated for 2 h at room temperature. Unbound tracer was removed by washing 5 times (each 1 ml) with washing solution (20 mM PBS, pH 7.4, 0.1% Triton X-100). Tube-bound chemiluminescence was measured by using the LB 953 (Berthold Technologies GmbH & Co. KG).

All antibodies were used in a sandwich immunoassay as coated tube and labelled antibody and combined in the following variations (see Table 2). Incubation was performed as described under hADM-Immunoassay. Results are given in ratio of specific signal (at 10 ng/ml ADM)/background (sample without ADM) signal.

TABLE 2
Signal to noise-ratio for anti-ADM antibody combinations

| Signal/ | NT-ADM | MR-ADM | CT-ADM |
|---|---|---|---|
| noise ratio | tracer | tracer | tracer |
| NT-ADM | / | 195 | 241 |
| MR-ADM | 204 | / | 904 |
| CT-ADM | 260 | 871 | / |

Surprisingly, we found the combination of MR-ADM and CT-ADM as combination for highest signal/noise ratio. This combination is measuring specifically bio-ADM (C-terminal amidated ADM), as the tracer antibody is directed against the C-terminal amidated end of bio-ADM.

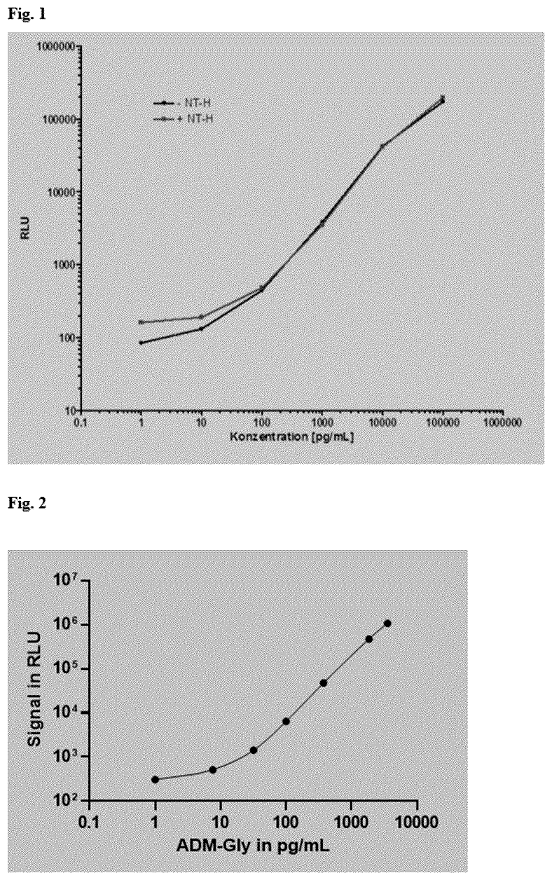

Subsequently, we used this antibody-combination for further investigations to measure bio-ADM. We used anti-MR-ADM as solid phase antibody and anti-CT-ADM as labelled antibody. A typical dose/signal curve is shown in FIG. 1. The analytical sensitivity (average of 10 runs, ADM-free sample +2SD) of the assay was 2 pg ADM/ml.

### Example 3—Measurement of Biomarkers

### 3.1. Measurement of Bio-ADM

Quantification of bio-ADM was conducted using the sphingotest bio-ADM assay as described elsewhere (Weber, J. et al. Sandwich Immunoassay for Bioactive Plasma Adrenomedullin. J. Appl. Lab. Med. An AACC Publ. 2, 222-233 (2017)) using the antibody combination as described in Example 2. Briefly, 96-well high binding polystyrene microtiter plates (Greiner Bio-One International AG) were coated (18 h at 20° C.) with monoclonal anti-ADM antibody, directed towards amino-acids 21-32 (SEQ ID NO: 10) of bio-ADM (anti-MR-ADM AK; 1 μg/0.2 mL per well in 50 mM Tris-HCl, 100 mM NaCl, pH 7.8). After blocking with 30 g/L Karion, 5 g/L BSA (protease free), 6.5 mmol/L monopotassium phosphate, 3.5 mmol/L sodium dihydrogen phosphate (pH 6.5), the plates were vacuum-dried.

100 μL of samples/calibrators were pipetted into coated microtiter plates. Afterwards 150 μL of MACN labelled tracer antibody (anti-CT-ADM AK; directed towards the amidated C-terminus of bio-ADM; SEQ ID NO: 11) the microtiter plates were incubated for 1 h at 22° C. under agitation at 600 rpm. Unbound tracer was removed by washing 5 times (each 350 μL per well) with washing solution (20 mM PBS, 1 g/L Triton X-100, pH 7.4). Well-bound chemiluminescence was measured for 1 s per well by using the Centro LB 960 microtiter plate luminescence reader (Berthold Technologies). The assay was calibrated using dilutions of synthetic human bio-ADM (American Peptide Company). The lowest calibrator did not contain bio-ADM, but a concentration of 2 μg/mL was assigned to facilitate logarithmic evaluation. The calibrators were lyophilized in 20 mmol K₂PO₄, 6 mmol/L Na-EDTA, 5 g/L BSA, 100 mol/L leupeptin, 50 mol/L amastatin, 10 μg/mL of anti-N-terminal ADM antibody (SEQ ID NO: 9), pH 8.0, and reconstituted in H₂O before use. A typical standard curve is shown in FIG. 1.

Healthy subjects (n=100, average age 56 years) were measured using the bio-ADM assay. The median value was 13.7 pg/ml, the lowest value 11 pg/ml and the 99^{th} percentile 43 pg/ml. Since the assay sensitivity was 2 pg/ml, 100% of all healthy subjects were detectable using the described bio-ADM assay.

### 3.2. Measurement of ADM-Gly

An antibody directed towards the glycine extended C-terminus of ADM-Gly (AK835/G4) using an immunization peptide according to SEQ ID NO: 12 was developed using the methods as described in Example 1.

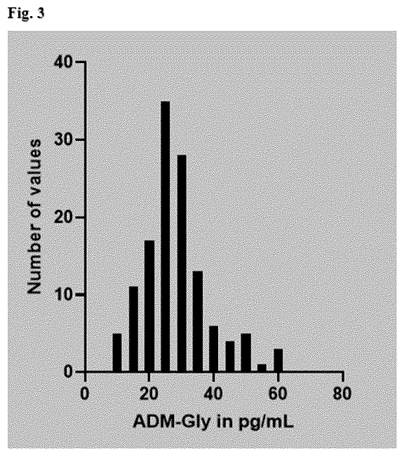

Quantification of ADM-Gly was conducted as follows: 96-well high binding polystyrene microtiter plates (Greiner Bio-One International AG) were coated (18 h at 20° C.) with monoclonal anti-ADM antibody, directed towards amino-acids 21-32 (SEQ ID NO: 10) of ADM-Gly (anti-MR-ADM-AK, 1 g/0.2 mL per well in 50 mM Tris-HCl, 100 mM NaCl, pH 7.8). After blocking with 30 g/L Karion, 5 g/L BSA (protease free), 6.5 mmol/L monopotassium phosphate, 3.5 mmol/L sodium dihydrogen phosphate (pH 6.5), the plates were vacuum-dried. 50 μL of samples/calibrators were pipetted into coated microtiter plates. Afterwards 200 μL of MACN labelled tracer antibody (AK835/G4, directed towards the glycine extended C-terminus of ADM-Gly, SEQ ID NO: 12). AK835/G4 had no cross-reactivity with bio-ADM) the microtiter plates were incubated for 18 h at 4° C. under agitation at 600 rpm. Unbound tracer was removed by washing 5 times (each 350 μL per well) with washing solution (20 mM PBS, 1 g/L Triton X-100, pH 7.4). Well-bound chemiluminescence was measured for 1 s per well by using the Centro LB 960 microtiter plate luminescence reader (Berthold Technologies). The assay was calibrated using dilutions of synthetic human ADM-Gly (Peptides and Elephants, Hennigsdorf, Germany). The lowest calibrator did not contain ADM-Gly, but a concentration of 1 μg/mL was assigned to facilitate logarithmic evaluation. The calibrators were lyophilized in 20 mmol K₂PO₄, 6 mmol/L Na-EDTA, 5 g/L BSA, 100 mol/L leupeptin, 50 gmol/L amastatin, 10 g/mL of anti-N-terminal ADM-antibody (SEQ ID NO: 9), pH 8.0, and reconstituted in H₂O before use. A typical standard curve is shown in FIG. 2. A normal distribution of ADM-Gly from n=128 self-reported healthy individuals is shown in FIG. 3. The median value was 27.1 pg/ml, the lowest value 10.1 pg/ml and the 97.5^{th} percentile 58.9 pg/ml.

### Example 4—Bio-ADM and ADM-Gly in Patients Intended to Receive CAR T-Cell Therapy

A cohort of seventeen patients with advanced relapsed and/or refractory lymphoma or leukemia was treated with CAR T-cells following a established procedure as described (Ayala Ceja et al., 2024). Blood samples were collected immediately prior to the administration of T-cells as well as after the treatment, triggered by clinical events including diagnosis and treatment of complications related to a cytokine release syndrome (CRS). CRS was defined and graded according to American Society for Transplantation and Cellular Therapy (ASTCT) guidelines (D. W. Lee et al., 2019). The grading system has defined a range of 5 grades, with grade 5 being the most severe condition defined as death due to CRS in which another cause is not the principle factor leading to this outcome. Blood samples for measurement of bio-ADM and ADM-Gly were gained just before the administration of T-cells in the frame of CAR T-cell therapy (baseline), and then at several time points in the days following the CAR T-cell therapy. Baseline concentrations of bio-ADM and ADM-Gly are shown in Table 3.

TABLE 3
Baseline bio-ADM- and ADM-Gly levels of patients
treated with CAR T-cells, and their CRS grading
resulting from the CAR T-cell therapy.

| Patient | bio-ADM | ADM-Gly | CRS |
|---|---|---|---|
| # | [pg/mL] | [pg/mL] | Grading |
| 1 | 54.3 | 40.7 | 2 |
| 2 | 10.4 | 27.4 | 3 |
| 3 | 114.5 | 336.5 | 2 |
| 4 | 17.0 | 66.5 | 1 |
| 5 | 26.2 | 41.7 | 1 |
| 6 | 08.3 | 18.9 | 0 |
| 7 | 12.5 | 21.7 | 2 |
| 8 | 35.8 | 101.6 | 2 |
| 9 | 16.3 | 37.4 | 0 |
| 10 | 15.6 | 30.5 | 0 |
| 11 | 96.1 | 85.3 | 3 |
| 12 | 51.5 | 142.4 | 3 |
| 13 | 155.8 | 192.0 | 4 |
| 14 | 17.5 | 46.7 | 0 |
| 15 | 14.8 | 48.0 | 0 |
| 16 | 32.4 | 37.1 | 3 |
| 17 | 14.4 | 41.2 | 3 |

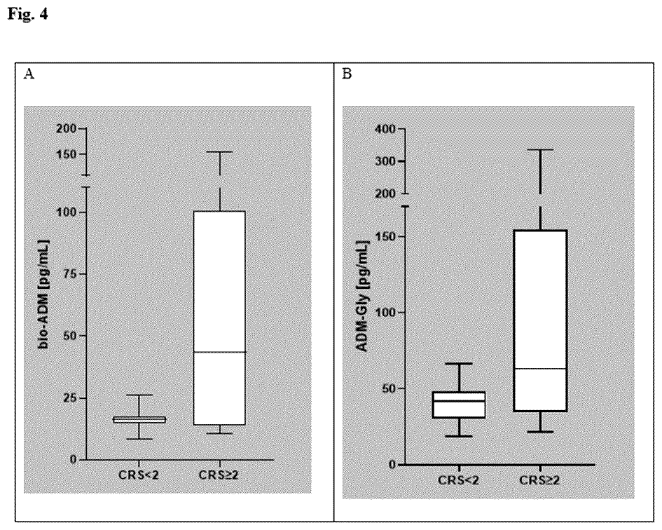

When the baseline concentrations of bio-ADM and ADM-Gly were separated in groups of patients who after the start of the CAR-T cell therapy either developed severe side-effects (defined as CRS score ≥2) or did not or only very moderately develop such effects (defined as CRS<2), it surprisingly turned out that patients developing severe side-effects presented with strongly elevated concentrations of bio-ADM and ADM-Gly already prior to start of the administration of T-cells in the frame of CAR T-cell therapy (median: 43.7 pg/ml and 63.3 pg/mL, respectively) compared to those patients who did not or only very moderately develop such effects (median: 16.3 pg/mL and 41.7 pg/mL, respectively) (FIG. 4). This observation demonstrates that measurement of bio-ADM, ADM-Gly or related peptides is suitable to detect prior to administration of T-cells in the frame of a CAR T-cell therapy the risk of development of severe side effects. Elevation of bio-ADM or ADM-Gly is considered an indication of endothelial dysfunction (van Lier et al., 2020). Thus, administration of an anti-ADM antibody suitable for the treatment of endothelial dysfunction in cancer patients with elevated levels of bio-ADM or ADM-Gly prior to start of administration of T-cells in the frame of a CAR T-cell therapy would be an ideal tool to prevent the development of severe side effects eventually developing after administration of CAR T-cells.

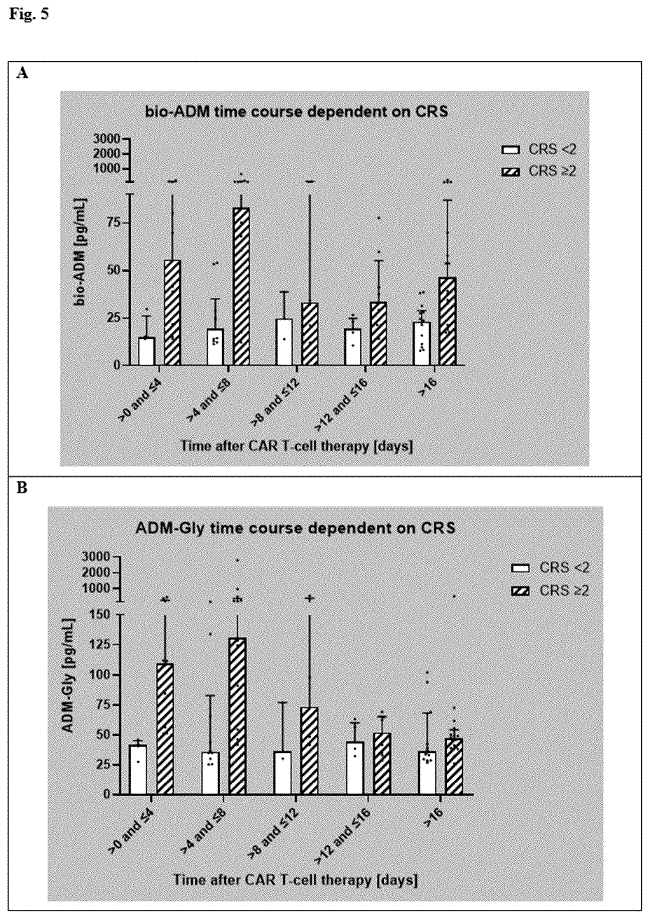

Both markers, bio-ADM and ADM-Gly, were also measured in the follow-up of the patients. As blood draws were not taken at the exact same time points after administration of CAR T-cells in the frame of CAR T-cell therapy for all patients, data from bio-ADM- and ADM-Gly measurements were grouped in time intervals of four days, e.g. >0 and ≤4 days, >4 and ≤8 days, >8 and ≤12 days, >12 and ≤16 days, >16 days, after administration of T-cells in the frame of CAR T-cell therapy. For both markers, bio-ADM and ADM-Gly, it was observed that concentrations increased after administration of T-cells in the frame of CAR T-cell therapy until the time interval >4 and ≤8 days, followed by a decline, only for patients who developed severe side-effects (defined as CRS score >2) (FIG. 5). In contrast, patients who did not or only very moderately develop such effects (defined as CRS<2), exhibited constant low levels of bio-ADM and ADM-Gly over the entire observation period. Thus, measurement of bio-ADM, ADM-Gly or related peptides is suitable to monitor endothelial dysfunction associated with the severity of side-effects arising from CAR T-cell therapy.

SEQUENCES

```
SEQ ID NO: 1-pre-proADM (aa 1-185)
                10 20 30 40 50
MKLVSVALMY LGSLAFLGAD TARLDVASEF RKKWNKWALS RGKRELRMSS
                60 70 80 90 100
SYPTGLADVK AGPAQTLIRP QDMKGASRSP EDSSPDAARI RVKRYRQSMN
              110 120 130 140 150
NFQGLRSFGC RFGTCTVQKL AHQIYQFTDK DKDNVAPRSK ISPQGYGRRR
              160 170 180
RRSLPEAGPG RTLVSSKPQA HGAPAPPSGS APHFL
SEQ ID NO: 2 (proADM: 164 amino acids (22-185 of preproADM))
                10 20 30 40 50 60
ARLDVASEFR KKWNKWALSR GKRELRMSSS YPTGLADVKA GPAQTLIRPQ DMKGASRSPE
                70 80 90 100 110 120
DSSPDAARIR VKRYRQSMNN FQGLRSFGCR FGTCTVQKLA HQIYQFTDKD KDNVAPRSKI
              130 140 150 160
SPQGYGRRRR RSLPEAGPGR TLVSSKPQAH GAPAPPSGSA PHFL
SEQ ID NO: 3-PAMP-Gly (aa 22-42 of pre-proADM SEQ ID NO: 1)
                10 20
ARLDVASEFR KKWNKWALSR G
SEQ ID NO: 4-PAMP-NH₂ (aa 22-42 of pre-proADM SEQ ID NO: 1)
                10 20
ARLDVASEFR KKWNKWALSR-NH₂
SEQ ID NO: 5-MR-proADM (aa 45-92 of pre-proADM SEQ ID NO: 1)
                10 20 30 40
ELRMSSSYPT GLADVKAGPA QTLIRPQDMK GASRSPEDSS PDAARIRV
SEQ ID NO: 6-ADM-Gly 1-53 (aa 95-147 of pre-proADM SEQ ID NO: 1)
                10 20 30 40 50
YRQSMNNFQG LRSFGCRFGT CTVQKLAHQI YQFTDKDKDN VAPRSKISPQ GYG
SEQ ID NO: 7-ADM 1-52-amide (aa 95-146 of pre-proADM SEQ ID NO: 1)
                10 20 30 40 50
YRQSMNNFQG LRSFGCRFGT CTVQKLAHQI YQFTDKDKDN VAPRSKISPQ GY-NH₂
SEQ ID NO: 8-CT-proADM (aa 148-185 of pre-proADM SEQ ID NO: 1)
                10 20 30 40
RRRRRSLPEA GPGRTLVSSK PQAHGAPAPP SGSAPHFL
SEQ ID NO: 9-ADM fragment 1-21 (aa 95-115 of pre-proADM SEQ ID NO: 1)
                10 20
YRQSMNNFQG LRSFGCRFGT C
SEQ ID No.: 10 (human ADM 21-32)
                10
CTVQKLAHQI YQ
SEQ ID No.: 11 (human ADM C-42-52)
                10
CAPRSKISPQ GY-CONH₂
SEQ ID No.: 12 (human ADM-Gly C-42-53)
                10
CAPRSKISPQ GYG
```

1. Method for the prediction of a risk of side effects and/or prediction of the severity of side effects in a cancer patient that is to be treated or has been treated with immune effector cells in the frame of an immune effector cell therapy (IECT), and/or for assessing an effect of a treatment of side effects in a cancer patient that has been treated with immune effector cells in the frame of an immune effector cell therapy (IECT), the method comprising the steps of:
determining the level of at least one endothelial dysfunction biomarker in a sample of bodily fluid of said patient, and
correlating said level of said at least one endothelial dysfunction biomarker in said sample to the risk of said side effects and/or the severity of said side effects in said patient and/or to the effect of said treatment of said side effects in said patient.

2. The method according to claim 1, wherein if said level of at least one endothelial dysfunction biomarker in a sample of bodily fluid obtained from said patient is above a threshold, said patient is at risk of developing said side effects and/or said treatment of side effects is being determined as being not effective.

3. The method according to claim 1, wherein said side effects are selected from the group comprising cytokine release syndrome (CRS), and immune effector cell-associated neurotoxicity syndrome (ICANS), preferably wherein the severity of said cytokine release syndrome (CRS) or immune effector cell-associated neurotoxicity syndrome (ICANS) is mild (grade 1), moderate (grade 2), severe (3) or life-threatening (grade 4) or leads to death (grade 5).

4. The method according to claim 1, wherein said immune effector cell therapy (IECT) is selected from the group comprising Chimeric Antigen Receptor T-cell (CAR-T) therapy, natural killer cell (NK) therapy, chimeric antigen receptor natural killer cell (CAR-NK) therapy, T cell receptor-engineered T-cell (TCR T) therapy, tumor-infiltrating T cell (TIT)), and cytokine-induced killer cell (CIK) therapy.

5. The method according to claim 1, wherein said endothelial dysfunction biomarker is selected from the group comprising Proadrenomedullin (proADM) or fragments thereof, adhesion molecules (e.g. E-selectin, P-selectin, ICAM-1, VCAM-1), molecules involved in the coagulation pathway (e.g. von Willebrand factor (vWF) and soluble thrombomodulin), endoglin, endocan, IL-6, CRP, LOX-1, CD40L, ADMA, proendothelin-1 or fragments thereof (in particular ET-1, Big-ET, NT-proET-1), matrix metalloproteinases (MMPs, e.g. MMP-7 and MMP-9), and tissue inhibitor of metalloproteinases (TIMPs, e.g. TIMP-1),
particularly selected from the group comprising proADM (SEQ ID NO. 2) or fragments thereof, adhesion molecules (e.g. E-selectin, P-selectin, ICAM-1, VCAM-1), molecules involved in the coagulation pathway (e.g. von Willebrand factor (vWF) and soluble thrombomodulin), endoglin, endocan, proendothelin-1 or fragments thereof (in particular ET-1, Big-ET, NT-proET-1), matrix metalloproteinases (MMPs, e.g. MMP-7 and MMP-9), and tissue inhibitor of metalloproteinases (TIMPs, e.g. TIMP-1).

6. The method according to claim 5, wherein said fragment of Pro-Adrenomedullin is selected from the group comprising, PAMP-Gly (SEQ ID NO: 3), mature PAMP (SEQ ID NO: 4), MR-proADM (SEQ ID NO: 5), ADM-Gly (SEQ ID NO: 6), mature ADM (SEQ ID NO: 7) and CT-proADM (SEQ ID NO: 8), preferably wherein said fragment of Pro-Adrenomedullin is MR-proADM and the threshold of the level of MR-proADM in a sample of bodily fluid of said patient is between 0.5 and 2 nmol/L, more preferred between 0.6 and 1.5 nmol/L, even more preferred between 0.7 and 1 nmol/L, most preferred said threshold is 0.8 nmol/L,
or said threshold is an x-fold of the median level of MR-proADM in a healthy population, in particular in the range between 1.2-fold and 4.9-fold, more preferred between 1.5-fold and 3.7-fold, even more preferred between 1.7-fold and 2.4-fold, most preferred said threshold is 2.0-fold of the median of the level of MR-proADM in a healthy population, or wherein said fragment of Pro-Adrenomedullin is mature PAMP and the threshold of the level of mature PAMP in a sample of bodily fluid of said patient is between 0.7 and 1.2 pmol/L, more preferred between 0.8 and 1.0 pmol/L, most preferred said threshold is 0.9 pmol/L,
or said threshold is an x-fold of the mean level of mature PAMP in a healthy population, in particular in the range between the 1.4-fold and 2.4-fold, more particular in the range between 1.6-fold and 2.0-fold, most particular said threshold is the 1.8-fold of the mean of the level of mature PAMP in a healthy population, or wherein said fragment of Pro-Adrenomedullin is PAMP-Gly and the threshold of the level of PAMP-Gly in a sample of bodily fluid of said patient is between 1.5 and 2.4 pmol/L, even more preferred between 1.8 and 2.2 pmol/L, most preferred said threshold is 2 pmol/L and 2.4 pmol/L, even more preferred between 1.8 and 2.2 pmol/L, most preferred said threshold is 2 pmol/L,
or said threshold is an x-fold of the mean level of PAMP-Gly in a healthy population, in particular in the range between the 1.3-fold and 2.1-fold, more particular in the range between 1.6-fold and 1.9-fold, most particular said threshold is the 1.7-fold of the mean of the level of PAMP-Gly in a healthy population, or wherein said fragment of Pro-Adrenomedullin is ADM-Gly and the threshold of the level of ADM-Gly in a sample of bodily fluid of said patient is between 25 and 125 pg/ml, more preferred between 30 and 100 pg/ml, even more preferred between 35 and 75 pg/ml, most preferred said threshold is 40 pg/ml,
or said threshold is an x-fold of the median level of ADM-Gly in a healthy population, in particular in the range between the 0.9-fold and 4.6-fold, more particular in the range between the 1.1-fold and 3.7-fold, more particular in the range between 1.3-fold and 2.8-fold, most particular said threshold is the 1.5-fold of the median of the level of ADM-Gly in a healthy population, or wherein said fragment of Pro-Adrenomedullin is mature ADM and the threshold of the level of mature ADM in a sample of bodily fluid of said patient is between 35 and 125 pg/ml, more preferred between 40 and 100 pg/ml, even more preferred between 50 and 90 pg/ml, most preferred said threshold is 70 pg/ml,
or said threshold is an x-fold of the median level of mature ADM in a healthy population, in particular in the range between the 2.6-fold and 9.1-fold, more particular in the range between the 2.9-fold and 6.3-fold, more particular in the range between 3.6-fold and 6.6-fold, most particular said threshold is the 5.1-fold of the median of the level of mature ADM in a healthy population, or wherein said fragment of Pro-Adrenomedullin is CT-proADM and the threshold of the level of CT-proADM in a sample of bodily fluid of said patient is between 75 and 350 pmol/L, more preferred between 100 and 250 pmol/L, even more preferred between 125 and 200 pmol/L, most preferred said threshold is 150 pmol/L,
or said threshold is an x-fold of the median level of CT-proADM in a healthy population, in particular in the range between the 1.0-fold and 4.5-fold, more particular in the range between the 1.3-fold and 3.2-fold, more particular in the range between 1.6-fold and 2.6-fold, most particular said threshold is the 1.9-fold of the median of the level of CT-proADM in a healthy population.

7. The method according to claim 1, wherein said sample of bodily fluid is selected from the group comprising whole blood, plasma and serum.

8. The method according to claim 1, wherein said determination of the level of at least one endothelial dysfunction biomarker and said correlation is performed more than once in said patient,
wherein particularly said determination of the level of at least one endothelial dysfunction biomarker and said correlation is performed once before administration of immune effector cells to said patient and one or more times after administration of immune effector cells to said patient; or
alternatively said determination of the level of at least one endothelial dysfunction biomarker and said correlation is performed at least twice after administration of immune effector cells to said patient.

9. The method according to claim 1, wherein for said patient that is to be treated with immune effector cells in the frame of an immune effector cell therapy (IECT) said determination of the level of at least one endothelial dysfunction biomarker and said correlation is carried out in the time span between lymphodepletion and administration of the immune effector cells, more particularly within 14 days, or within 7 days, or within 2 days before said immune effector cells are administered to said patient, even more particularly within 48 hours, or within 24 hours, or within 12 hours, or within 6, or within 2 hours, or within 1 hour before said immune effector cells are administered to said patient.

10. The method according to claim 1, wherein said patient that has been treated with immune effector cells has not developed side effects from said treatment at the time-point of taking said sample.

11. The method according to claim 1, wherein for said patient that has been treated with immune effector cells said determination of the level of at least one endothelial dysfunction biomarker and said correlation is carried out within 1 week, or within 2 days after said immune effector cells have been administered to said patient, even more particularly within 48 hours, or within 24 hours, or within 12 hours, or within 6, or within 3 hours after said immune effector cells have been administered to said patient.

12. The method according to claim 1, wherein said treatment of side effects is selected from the group comprising antihistamines, non-steroidal anti-inflammatory drugs (NSAIDs), immunosuppressive therapy, vasopressors, fluids and supportive oxygen supply, wherein preferably said immunosuppressive therapy is selected from the group comprising anti-IL-6 antibody (e.g. siltuximab), anti-IL-6 receptor antibody (e.g. tocilizumab), anti-TNF-antibody (e.g. etanercept, infliximab), anti-IL-1-antibody (e.g. anakinra), and/or corticosteroids.

13. The method according to claim 1, wherein said treatment of side effects is administered if an increased risk of said side effects is predicted for said patient and/or said patient is in need of adjustment of said treatment of side effects if said treatment of side effects is being determined as not effective.

14. Method for prevention or treatment of side effects of immune effector cell therapy (IECT) in a cancer patient that is to be treated or has been treated with immune effector cells in the frame of an immune effector cell therapy (IECT), wherein the treatment is immunosuppressive therapy, wherein said patient is selected by a method comprising the steps:
determining at least one endothelial dysfunction biomarker in a sample of bodily fluid obtained from said patient and
correlating said level of at least one endothelial dysfunction biomarker in said sample to said risk of side effects, and
wherein said side effects are selected from the group comprising cytokine release syndrome (CRS), and immune effector cell-associated neurotoxicity syndrome (ICANS).

15. Medicament for use in the prevention or treatment of side effects of immune effector cell therapy (IECT) in a cancer patient that is to be treated or has been treated with immune effector cells in the frame of an immune effector cell therapy (IECT), wherein said patient is selected by a method comprising the steps:
determining at least one endothelial dysfunction biomarker in a sample of bodily fluid obtained from said patient and
correlating said level of at least one endothelial dysfunction biomarker in said sample to said risk of side effects, and
wherein said side effects are selected from the group comprising cytokine release syndrome (CRS), and immune effector cell-associated neurotoxicity syndrome (ICANS), and wherein said medicament is an immunosuppressive medicament.

16. A method for patient stratification and/or selection of a patient for treatment of side effects, wherein said patient is a cancer patient that is to be treated or has been treated with immune effector cells in the frame of an immune effector cell therapy (IECT), said method comprising:
determining the level of at least one endothelial dysfunction biomarker in a sample of bodily fluid of said patient and
assigning if said patient is to receive treatment of said side effects based on said level of at least one endothelial dysfunction biomarker in said sample.
